(19) United States
(12) Patent Application Publication
(10) Pub. No.: US 2016/0122402 A1
(43) Pub. Date: May 5, 2016

(54) RETRO POLYPEPTIDES FOR ACTIVATION IMMUNITY TO CANCER AND VIRAL INFECTION

(71) Applicant: IMPERIAL INNOVATIONS LIMITED, London (GB)

(72) Inventor: Philip ASHTON-RICKARDT, London (GB)

(21) Appl. No.: 14/893,482

(22) PCT Filed: May 23, 2014
(86) PCT No.: PCT/GB2014/051603

§ 371 Date: Nov. 23, 2015

(30) Foreign Application Priority Data
May 24, 2013 (GB) 1309421.4

(51) Int. Cl. C07K 14/47; G01N 33/50; C12N 15/113

(52) U.S. Cl. CPC C07K 14/47; C12N 15/113; G01N 33/505; A61K 38/00

(57) ABSTRACT

A polypeptide that drives T cell proliferation and differentiation, and methods and agents for modulating the expression and/or function of the polypeptide. Agents that up regulate the expression and/or function of the polypeptide are useful in the treatment or prevention of diseases and disorders that would benefit from stimulation of T cell proliferation and/or differentiation, such as cancer and chronic viral infections. Agents that down-regulate the expression and/or function of the polypeptide are useful in the treatment or prevention of diseases, disorders and conditions involving unwanted or excessive proliferation and/or differentiation of T cells, such as autoimmune and inflammatory diseases. Isolated T cells containing an expression vector encoding a polypeptide is also disclosed. Such T cells may be used in adoptive cell transfer therapies.

The present invention relates to polypeptides which drive T cell proliferation and differentiation, nucleic acids encoding the polypeptides and methods involving the polypeptides and/or the nucleic acid.

### BACKGROUND

Using the immune system to destroy cancer cells that are resistant to traditional cancer therapies has been a goal for some time. Cytotoxic T Lymphocytes (CTLs) directly kill tumour cells and so are important cell targets for cancer immunotherapy. A major stumbling block to cancer immunotherapy is the suppression of tumour cell killing in cancer. Therapies that either positively stimulate or inhibit negative regulation of tumour cell killing result in partial immunity to solid tumours.

Efforts to harness the power of cellular immunity based on T lymphocytes against cancer are only now beginning to bear fruit. Most T lymphocytes that react against cancer cells are specific for peptides that are also expressed by normal cells (self-peptides) [1]. Immunological tolerance either eliminates or functionally inactivates T cells that are reactive to self-peptides and so protects against autoimmune disease [2]. Current approaches to T cell immunity to cancer include:
Activating Cytokines: Interferon-alpha 2b (IF-α2b) and IL-2 have been approved for the adjuvant treatment of solid tumours [3, 4].
Blocking Inhibitory Cytokines: IL-10 produced by macrophages or melanoma cells directly favours tumour growth and suppresses T cell immunity [5].
Stimulating Co-Receptors: CD40 is co-receptor required for the generation of fully functional CTL and when stimulated with agonist antibody removes the need for CD4 T cell help and can break tolerance to tumour antigens [6-8].
Vaccination: Vaccination with peptide epitopes has been broadly tested in the clinic for over 16 years with very low response rates [9, 10]
Blocking Negative Regulators: Co-inhibitory receptors, which are expressed on T cells and receive inhibitory signals from tolerogenic DC, represent an important class of negative regulators [11, 12]. The CTLA4-blocking antibody Ipilimumab was approved (as Yervoy) by the FDA in 2011.
Adoptive Cell Therapy of T cells: Adoptive cell transfer (ACT) strategies are based on expanding large quantities of tumour-specific T cells ex vivo and their re-infusion into patients. [13-15]. ACT of T cell receptor (TCR) engineered lymphocytes leads to tumour responses in patients with metastatic melanoma [16, 17].

To date, three agents, which act directly and primarily on T lymphocytes, have been approved for clinical use against malignant melanoma (IL-2, ipilimumab and sipuleucel-T). However each therapy gives a relatively modest increase in patient survival. In addition, combination immunotherapy (e.g. gp100 vaccination and CTLA-4 blockade) does not always increase efficacy [18]. Therefore cancer immunotherapy would benefit from the targeting of new pathways to increase the level of anti-tumour CTLs.

Chronic viral infections, such as those caused by the human immunodeficiency virus (HIV) and the hepatitis B and C viruses (HBV, HCV) affect more than 500 million people worldwide. Available options to prevent and treat chronic viral infections are unsatisfactory. Vaccines do not provide immunity in the chronically virally infected and chemical inhibitors require life-long treatment.

Using the immune system to kill viruses is a major goal of immunotherapy and vaccination. A major obstacle is the suppression of virus killing through immune exhaustion of lymphocytes, which results in chronic viral infection.

Both CTLs [19] and antibodies [20] control persistent viral infection and so new immunotherapies would benefit from approaches that activate both.

### SUMMARY OF THE INVENTION

In a first aspect, the invention provides an isolated polypeptide comprising: (a) the amino acid sequence of SEQ ID NO: 1 or an amino acid sequence having at least 70% identity to SEQ ID NO: 1, or (b) the amino acid sequence of SEQ ID NO: 2, or an amino acid sequence having at least 70% identity to SEQ ID NO: 2.

A second aspect of the invention provides an expression vector comprising a nucleic acid encoding the polypeptide of the first aspect.

In a third aspect, the invention provides an in vitro method of increasing the proliferation of a T cell population, comprising transducing at least one T cell in the population with the vector of the second aspect of the invention.

In a fourth aspect, the invention provides an isolated T cell containing the expression vector of the third aspect.

In a fifth aspect, the invention provides a method of identifying an agent which modulates the binding affinity of the polypeptide of the first aspect of the invention for a target, comprising:
i) contacting the polypeptide or a biologically active fragment thereof with the target and a candidate agent;
ii) measuring the affinity of the polypeptide or biologically active portion thereof for the target; and
iii) comparing the affinity of the polypeptide or biologically active fragment thereof for the target in the presence of the candidate agent with the affinity of the polypeptide or biologically active portion thereof for the target in the absence of the candidate compound,
   wherein an agent which modulates the binding affinity of the polypeptide is identified if the affinity is increased or decreased in the presence of the agent compared to in the absence of the agent.

### DESCRIPTION OF THE FIGURES

FIG. 1A: Human Retro polypeptide sequence Q32CV2 (SEQ ID NO: 1).

FIG. 1B: Human Retro cDNA sequence (SEQ ID NO: 3).

FIG. 2A: Mouse Retro polypeptide sequence A2AVQ5 (SEQ ID NO: 2).

FIG. 2B: Mouse Retro cDNA sequence (SEQ ID NO: 4).

FIG. 3: Hypothesis for how Retro induces CTL proliferation.

FIG. 4: Phenotype of Retro homozygous mutant mice.

FIG. 5A: CD8⁺ T cell intrinsic effect of Retro mutation.

FIG. 5B: Retro-mutant CTLs are hyper-proliferative in vivo.

FIG. 6: The ENU-induced mutation that correlates with the Retro phenotype.

FIG. 7: CTLL-2 cells with Retro genes.

FIG. 8: Retro mRNA levels for CTL in vitro.

FIG. 9: Retro mRNA levels for CTL in vivo.

FIG. 10A: CD8 T cells from Retro mutant mice were hyper-proliferative as evidence by BrdU⁺ incorporation.

FIG. 10B: Retro mutant CTL transduced by Retro shRNA show reduced proliferation relative to CTL transduced with scrambled shRNA.

FIG. 10C: Retro mRNA levels reduced using Retro shRNA.

FIG. 11: Retro mutant mice have increased CTL immunity to melanoma.

FIG. 12: Affinity of Retro binding to RNA containing an ARE motif.

FIG. 13: A Retro protein vector sequence (SEQ ID NO: 5), protein sequence in bold.

FIG. 14: Plasmid map of a Retro protein expression vector.

FIG. 15: 6×His-TEV-Retro(1-418)_optEC protein sequence (SEQ ID NO: 6)

FIG. 16: Chromatogram of Ni-NTA purification of Retro protein in the unfolded state.

FIG. 17: SDS-PAGE and anti-His Western blot of Ni-NTA purified samples.

FIG. 18: SDS-PAGE analysis of Retro protein refolded in a refolding screen (XTAL Biostructures, Inc).

FIG. 19: Over expression of hRetro increases expansion of Jurkat T cells.

FIG. 20: Increased development of primary and memory CD8 T cells after vaccination of Retro mutant mice.

FIG. 21: In vivo validation of the causative Retro mutation.

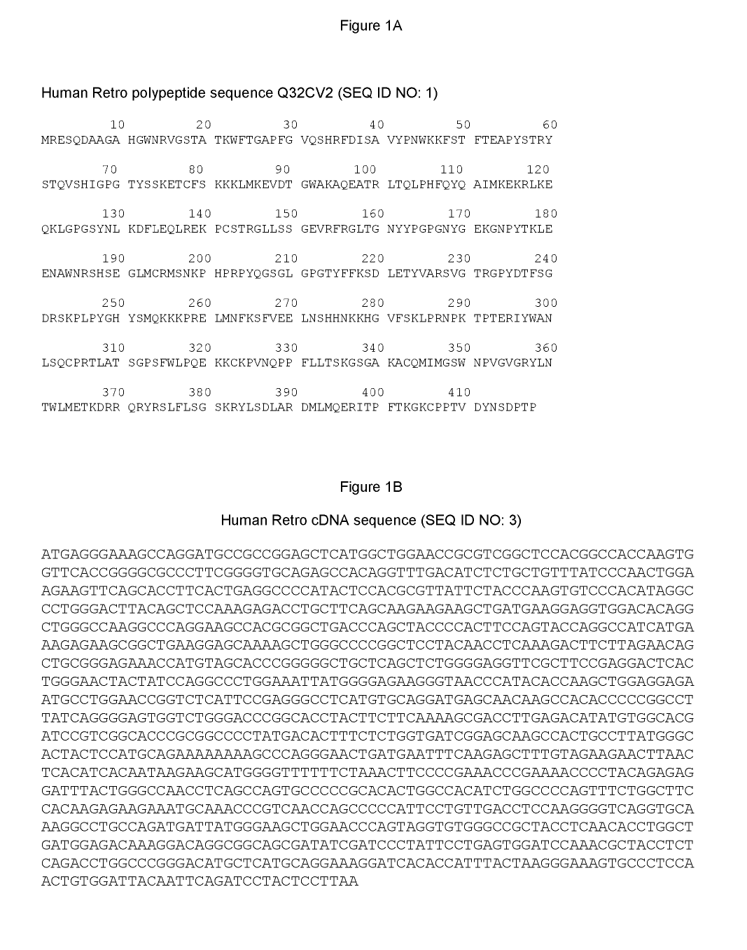

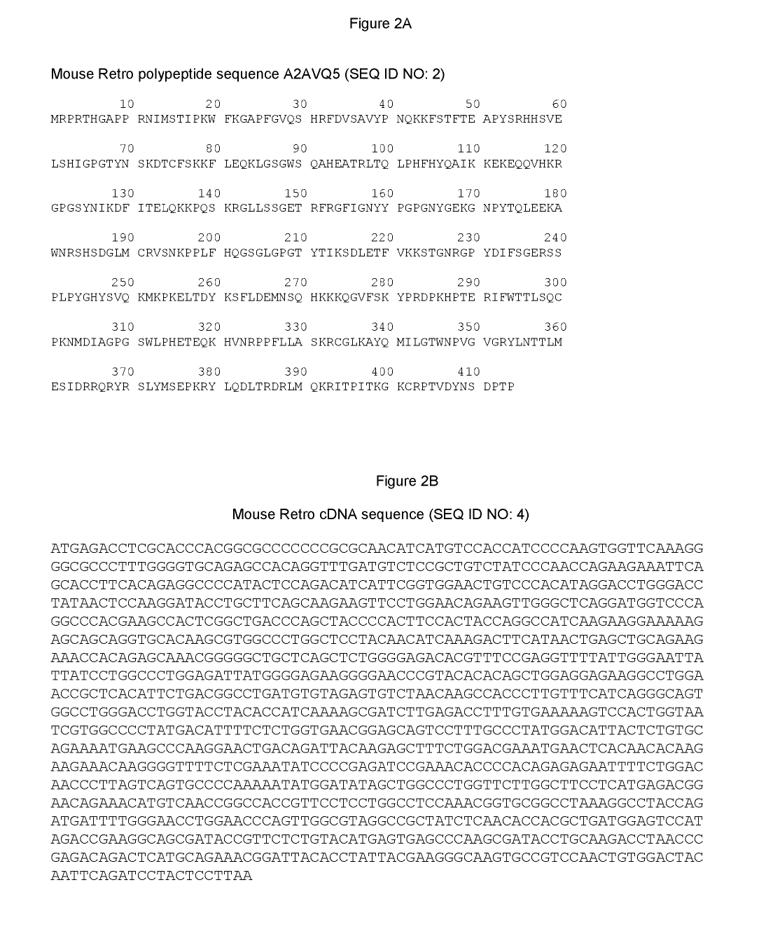

### DETAILED DESCRIPTION OF THE INVENTION

The present inventors have discovered a target and pathway for the activation of immunity to cancer and viral infection. The polypeptides of the present invention can drive T cell proliferation and differentiation, in particular Cytotoxic T lymphocyte (CTL) proliferation and differentiation.

The gene encoding one polypeptide of the invention was discovered using an unbiased forward genetic approach to identify mouse mutants with CTLs that are resistant to immunosuppression (see Example 1 for further details). The gene was designated “Retro”. The term “Retro” is used herein to refer to polypeptides of the invention (including both the mouse and human polypeptides) and nucleic acids encoding such polypeptides (including both the mouse and human genes). It will be apparent to the skilled person which is meant from the context in which the term is used.

As is discussed and demonstrated further in the Examples, wild-type Retro promotes the proliferation of CTLs. The present inventors have also discovered that an E50G mutation in the mouse Retro gene (equivalent to an E53G mutation in the human gene) is a gain-of-function mutation which gives rise to increased CTL proliferation (a hyper-proliferation phenotype) relative to that elicited by the wild type gene. Furthermore, it has been discovered that increased expression of Retro mRNA contributes to the phenotype of CTL hyper-proliferation.

Linear homology searches (e.g. BLAST) indicate that Retro (mouse-BC055111/humanC1ORF177; Swiss Prot: A2AVQ5) has no homology to any other known protein or family of proteins. The only homologues are C1ORF177 orthologues, which are conserved from vertebrates through to sponges. The lack of any signal sequences or membrane spanning domain and the relative positive charge (mw=48 kD, PI=9.8), predicts that Retro is a cytosolic/nuclear protein that binds nucleic acid (PROPSEARCH). It is unlikely that Retro is a transcription factor because it has no homology to any domains used by transcription factors. The web-based method “FuncBase” generated functional linkage for C1ORF177 based on quantitative annotations of genes with Gene Ontogeny (GO) terms and revealed interaction with RNA-binding proteins (RNA-BP) [21].

RNA-BPs regulate mRNA turnover and protein translation through binding to sequence elements in the 3-untranslated region (3′-UTR) [22]. Examples of these 3′-UTR sequence motifs include the AU-rich element (ARE -5′-UAUUUAU-3′ core sequence) [23], and HuD (motifs 5′-UUUUUAAA-3′ and 5′-UUUCUUU-3′) [24]. The BCNET mutual information algorithm was used to identify likely gene targets for Retro in human gene networks [25, 26]. Several genes interacting with Retro harboured one or more ARE or HuD motifs and encoded proteins that control cell proliferation (e.g. methylene tetrahydrofolate reductase [MTHFR] and large tumour suppressor, homolog 1 [LATS1]). In addition, several of the gene targets (e.g. KLHL30-AS1 and LOC389247) encode antisense mRNA, which is consistent with Retro acting as an RNA-BP. Filter-binding assays revealed that affinity (Kd) of binding between recombinant mouse Retro and an oligoribonucleotide containing an ARE motif was about 153 nM (see Examples).

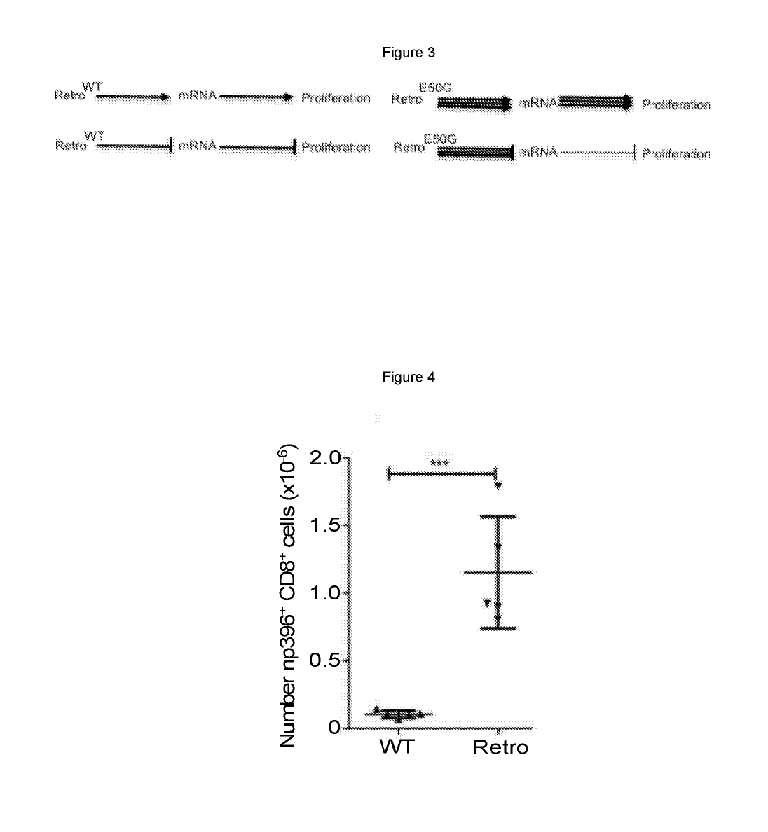

Without wishing to be bound by theory, it is believed that, in vivo, Retro may increase CTL proliferation either by binding and stabilising pro-proliferative mRNA, or by binding and destabilising anti-proliferative mRNA. FIG. 3 shows possible mechanisms of action: Wild-type Retro binds to the 3′-UTR of mRNA (arrow in FIG. 3) to increase the expression of genes that drive (arrow) the proliferation and/or differentiation of CTLs. Another possible mechanism of action is that wild type Retro binds to 3′-UTR of mRNA (blocked line) to decrease the expression of genes that block (blocked line) the proliferation and/or differentiation of CTLs. E50G Retro has increased affinity for 3′-UTR of mRNA which results in the super expression of pro-proliferation genes or super-repression of anti-proliferation genes. This results in more proliferation either by direct activation or by decreased repression.

The invention provides an isolated polypeptide comprising an amino acid sequence having at least 70% identity to SEQ ID NO: 1 (the wild type human Retro polypeptide sequence). The polypeptide may comprise an amino acid sequence which is 75%, 80%, 85%, 90%, 95%, 96%, 97%, 98%, 99% or 100% identical to the sequence of SEQ ID NO: 1. Such a polypeptide may have a mutation at position 53 relative to the wild type human Retro sequence (glutamate). The residue at position 53 of the mutated human Retro sequence may be any small non-polar amino acid. Examples include glycine, alanine, valine, leucine and isoleucine. Preferably the residue is glycine or alanine.

In some embodiments, the polypeptide of the invention comprises or consists of a sequence that is 100% identical to SEQ ID NO: 1 except that the glutamate residue at position 53 is replaced with a glycine residue (an “E53G” mutation”).

The invention also provides an isolated polypeptide comprising an amino acid sequence having at least 70% identity to SEQ ID NO: 2 (the wild type mouse Retro polypeptide sequence). The polypeptide may comprise an amino acid sequence which is 75%, 80%, 85%, 90%, 95%, 96%, 97%, 98%, 99% or 100% identical to the sequence of SEQ ID NO: 2. Such a polypeptide may have a mutation at position 50 relative to the wild type mouse Retro sequence (glutamate). The residue at position 50 of the mutated mouse Retro sequence may be any small non-polar amino acid. Examples include glycine, alanine, valine, leucine and isoleucine. Preferably the residue is glycine or alanine.

In some embodiments, the polypeptide of the invention comprises or consists of a sequence that is 100% identical to SEQ ID NO: 2 except that the glutamate residue at position 50 is replaced with a glycine residue. The “E50G” mutation increases the affinity of Retro for RNA species comprising the ARE motif. This may contribute to the physiological effect of the mutant polypeptide.

The “percent identity” of two polypeptide or amino acid sequences or two nucleic acid sequences can be or is generally determined by aligning the sequences for optimal comparison purposes (e.g., gaps can be introduced in either sequences for best alignment with the other sequence) and comparing the nucleotides at corresponding positions. The “best alignment” is an alignment of two sequences that results in the highest percent identity. The percent identity is determined by the number of identical nucleotides in the sequences being compared (i.e., % identity=# of identical positions/total # of positions×100). The determination of percent identity between two sequences can be accomplished using a mathematical algorithm known to those of skill in the art.

The polypeptide of the first aspect of the invention may have one or more substitutions, insertions, additions or deletions relative to the wild type sequences (SEQ ID NO: 1 and SEQ ID NO: 2). Preferably, the number of substitutions, insertions, additions or deletions is limited, i.e. no more than 1, 2, 3, 4, 5, 6, 7, 8, 9, 10 substitutions, no more than 1, 2, 3, 4, 5, 6, 7, 8, 9, 10 insertions, no more than 1, 2, 3, 4, 5, 6, 7, 8, 9, 10 additions, and no more than 1, 2, 3, 4, 5, 6, 7, 8, 9, 10 deletions compared to the wild type sequences (SEQ ID NO: 1 and SEQ ID NO: 2).

The polypeptides or fragments thereof of the present invention may be provided in isolated or recombinant form, and may be fused to other moieties. The polypeptides or fragments thereof may be provided in substantially pure form, that is to say free, to a substantial extent, from other proteins. Thus, a polypeptide may be provided in a composition in which it is the predominant component present (i.e. it is present at a level of at least 50%; preferably at least 75%, at least 90%, or at least 95%; when determined on a weight/weight basis excluding solvents or carriers).

In certain embodiments, the polypeptides of the invention comprise additional amino acid sequences, for example sequences fused to the N and/or C terminus of the polypeptide. Additional N-terminal or C-terminal sequences may be provided for various reasons. Techniques for providing such additional sequences are well known in the art. Additional sequences may be provided in order to alter the characteristics of a particular polypeptide. This can be useful in improving expression or regulation of expression in particular expression systems. For example, an additional sequence may provide some protection against proteolytic cleavage. This has been done for the hormone Somatostatin by fusing it at its N-terminus to part of the β galactosidase enzyme (Itakwa et al., Science 198: 105-63 (1977)).

Additional sequences can also be useful in altering the properties of a polypeptide to aid in identification or purification. For example, a fusion protein may be provided in which a polypeptide is linked to a moiety capable of being isolated by affinity chromatography. The moiety may be an antigen or an epitope and the affinity column may comprise immobilised antibodies or immobilised antibody fragments which bind to said antigen or epitope (desirably with a high degree of specificity). The fusion protein can usually be eluted from the column by addition of an appropriate buffer.

N-terminal or C-terminal sequences may be of the type generally referred to as “tags”. Such tags may be used to identify, isolate and/or purify the polypeptide. Non-limiting examples include, in general, affinity tags, solubilisation tags, chromatography tags and epitope tags. Non-limiting examples of specific tags include FLAG-tag, MYC-tag, HA-tag, GST-tag, Strep-tag and His-tag. The skilled person will be aware of many other general classes of tags and more specific examples of each class.

Additional N-terminal or C-terminal sequences may, however, be present simply as a result of a particular technique used to obtain a polypeptide and need not provide any particular advantageous characteristic to the polypeptide. Such polypeptides are within the scope of the present invention. Whatever additional N-terminal or C-terminal sequence is present, it is preferred that the resultant polypeptide should exhibit the biological activity of the polypeptides of the invention.

One preferred example of an affinity tag is an N-terminal TEV protease cleavable 6×His-tag. An advantage of a 6×His-tag is that it facilitates purification and does not interfere with binding assays involving the polypeptide such as thermal shift assays.

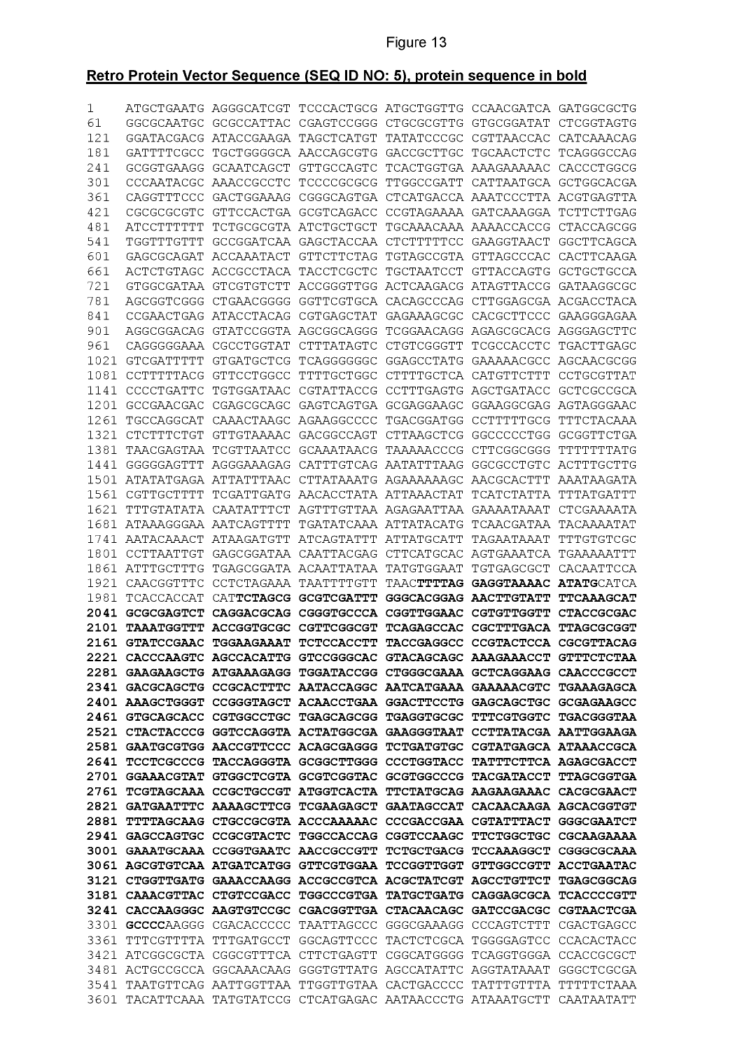

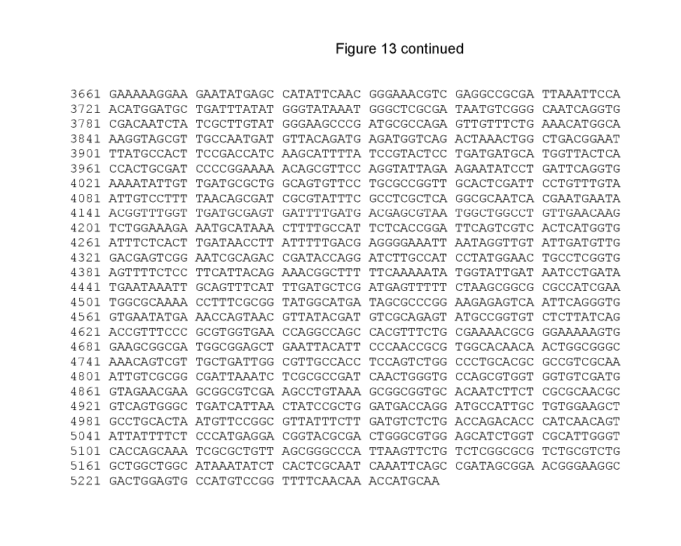

In order to optimise expression of a Retro polypeptide of the invention in a host cell, codon usage in nucleic acid encoding the polypeptide may be optimised for that host cell. The host cell may be E. coli. For example, the nucleic acid sequence shown in FIG. 13 (SEQ ID NO: 5) is optimised for expression in E coli.

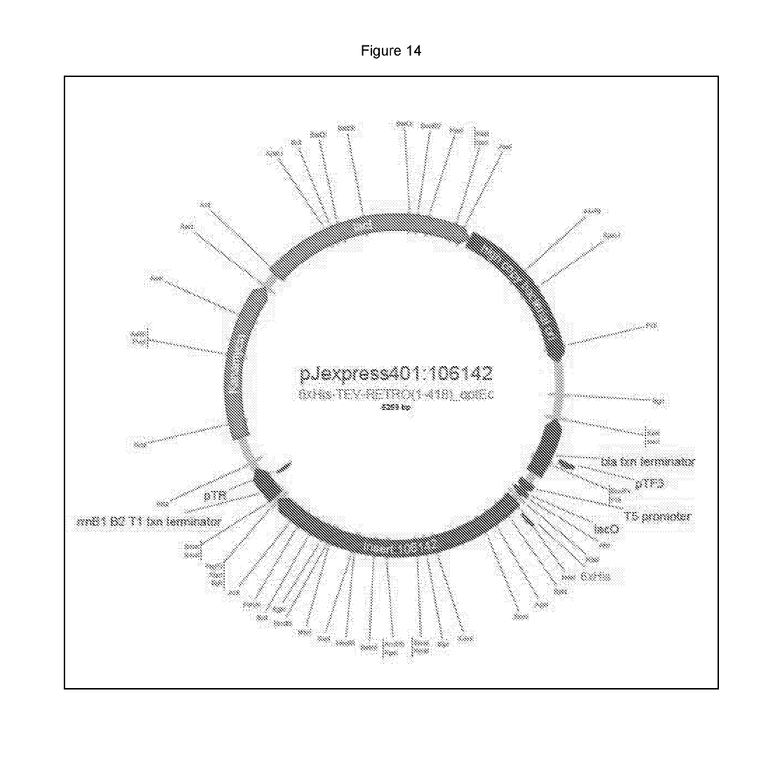

The second aspect of the invention provides an expression vector comprising nucleic acid encoding a polypeptide of the first aspect. In some embodiments, the vector comprises a nucleic acid sequence encoding the wild type human Retro polypeptide (SEQ ID NO: 3). In other embodiments, the vector comprises a nucleic acid sequence encoding the wild type mouse Retro polypeptide (SEQ ID NO: 4). The vector may comprise a nucleic acid encoding a Retro polypeptide which includes one or more mutations. The one or more mutations may comprise an E53G mutation in a human Retro polypeptide sequence. The one or more mutations may comprise an E50G mutation in a mouse Retro polypeptide sequence. In one embodiment, the vector may comprise the nucleic acid shown in SEQ ID NO: 5, which encodes the 6×His-TEV-Retro(1-418)_optEC polypeptide sequence shown in SEQ ID NO: 6. FIG. 14 shows a map of one Retro expression vector falling within the scope of the present invention. Expression of a Retro polypeptide in E. coli using this vector is discussed in the Examples.

As used herein with respect to nucleic acid molecules, “isolated or “recombinant” means any of a) amplified in vitro by, for example, polymerase chain reaction (PCR), b) recombinantly produced by cloning, c) purified by, for example, gel separation, or d) synthesised, such as by chemical synthesis.

The nucleic acid, for example DNA and RNA, may be synthesised using methods known in the art, such as using conventional chemical approaches or polymerase chain reaction (PCR) amplification. Nucleic acid molecules encoding polypeptides of the present invention also permit the identification and cloning of the Retro gene, for instance by screening cDNA libraries, genomic libraries or expression libraries.

A variety of host-expression vector systems may be utilised to express a polypeptide of the invention. Such host-expression systems represent vehicles by which the coding sequences of interest may be produced and subsequently purified, but also represent cells which may, when transformed, transduced or transfected with the appropriate nucleotide coding sequences, express the polypeptide of the invention in situ. These include but are not limited to microorganisms such as bacteria (e.g., E. coli, B. subtilis) transformed with recombinant bacteriophage DNA, plasmid DNA or cosmid DNA expression vectors containing polypeptide coding sequences; yeast (e.g., Saccharomyces, Pichia) transformed with recombinant yeast expression vectors containing polypeptide coding sequences; insect cell systems infected with recombinant virus expression vectors (e.g., baculovirus) containing the polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing polypeptide coding sequences; or mammalian cell systems (e.g., COS, CHO, BHK, 293, 3T3 cells) harbouring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the polypeptide being expressed. For example, when a large quantity of such a polypeptide is to be produced, vectors which direct the expression of high levels of fusion protein products that are readily purified may be desirable. Such vectors include, but are not limited, to the E. coli expression vector pUR278 (Ruther et al., 1983, EMBO J. 2:1791), in which the polypeptide coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The polypeptide coding sequence may be cloned individually into non-essential regions (for example, the polyhedrin gene) of the virus and placed under control of an AcNPV promoter (for example, the polyhedrin promoter). In mammalian host cells, a number of viral-based expression systems (e.g., an adenovirus expression system) may be utilised.

As discussed above, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of polypeptides of the invention, stable expression is preferred. For example, cells lines that stably express the polypeptide can be produced by transfecting the cells with an expression vector comprising the nucleotide sequence of the polypeptide and the nucleotide sequence of a selectable (e.g., neomycin or hygromycin), and selecting for expression of the selectable marker. Such engineered cell lines may be particularly useful in screening and evaluation of compounds that interact directly or indirectly with the polypeptide.

The expression levels of the polypeptide of the invention can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing the polypeptide is amplifiable, increase in the level of inhibitor present in culture of host cell will increase the number of copies of the marker gene. Since the amplified region is associated with the polypeptide gene, production of the polypeptide will also increase (Crouse et al., 1983, Mol. Cell. Biol. 3:257).

Once the polypeptide of the invention has been recombinantly expressed, it may be purified by any method known in the art for purification of a polypeptide, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilising an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni²⁺ nitriloacetic acid-agarose columns and histidine-tagged proteins are selectively eluted with imidazole-containing buffers.

In certain aspects of the invention, the vector may be for use in the treatment and/or prophylaxis of cancer or chronic viral infection.

The vectors of the present invention may be integrating or non-integrating vectors. The vectors may be selected from the group comprising retroviruses, lentiviruses, adenoviruses, adeno-associated viruses, non-viral vectors and combinations of these vectors.

Retroviruses may be selected from murine leukaemia virus (MLV), mouse mammary tumour virus (MMTV), Rouse sarcoma virus (RSV), Moloney murine leukaemia virus (MoMLV), Fujinami sarcoma virus (FuSV), Moloney murine sarcoma virus (Mo-MSV), Abelson murine leukaemia virus (A-MLV) and Avian erythroblastoma virus (AEV).

Lentiviruses may be selected from human immunodeficiency virus (HIV), simian immunodeficiency virus (SIV), feline immunodeficiency virus (FIV), equine infectious anaemia virus (EIAV), caprine arthritis encephalitis virus (CAEV), bovine immunodeficiency virus (BIV) and Jembrana disease virus (JDV) based vectors.

Adenoviruses may be selected from adenovirus type 5 first and second generation and gutless vectors. Details of adenovirus can be found GenBank accession number M73260. Adeno-associated viruses may be selected from all adeno-associated serotypes.

Retroviruses integrate into host cell DNA and have the potential for lifelong expression. However, retroviruses can potentially cause insertional mutagenesis due to insertion into the host's chromosomes. Lentiviruses can also integrate into the host's DNA. As with retroviruses, lentiviruses can potentially cause mutations when they are inserted into the host's chromosomes. Adeno-associated viruses can also be integrated into the host's DNA albeit to a lesser extent than retroviruses. Retroviruses, lentiviruses and adeno-associated viruses therefore have potential for long term expression in the host.

Adenoviruses can achieve transgene expression at high levels. However, they are usually non-integrating vectors and therefore do not insert themselves into the host's genome and accordingly have to be repeatedly administered in gene therapy applications.

Suitable retroviruses and lentiviruses for use in the present invention may be obtained from Coffin et al (“Retroviruses” 1997 Cold Spring Harbour Laboratory Press Eds: J M Coffin, S M Hughes, H E Varmus pp 758-763). Details on the structure of the genome of retroviruses are well known and may be found in the art. Details concerning lentiviruses are well known and may also be found in the art. For example, details on HIV may be found from the NCBI Genbank (ie. Genome Accession No AF033819), details on EIAV may be found from ICTVdB—The Universal Virus Database, version 3. http/www.ncbi.nlm.nlh.gov/ICTVdb/ICTVdB/. (accession number 00.061.1.06.003), and details on FIV may be found from the ICTVdB (accession number 00.061.1.06.004). In addition, details concerning adenovirus type 5 may be found from the NCBI Genbank (ie. Genome Accession No. M73260). Details concerning adeno-associated virus type 2 may be found from the ICTVdB Virus accession number: 50103001.

Viral vectors have a natural tropism for certain organs and are otherwise efficient mediators of nucleic acid molecule delivery. In the case of cancer, viral vectors have usually been administered by intratumoural injection. In the case of viral nucleic acid molecule delivery, the nucleic acid construct may be devised with a nucleic acid molecule encoding a polypeptide of the first aspect included as appropriate for the virus type being used in the delivery process.

Non-viral vectors may be selected from all vectors that do not integrate into host chromosomes. Non-viral vectors can either be physical in character (e.g. hydrodynamics, electroporation, biolistics, injection etc.) or synthetic (e.g. cationic liposome/micelle-based or cationic polymer-based). Physical vectors may be designed essentially for local/regional delivery only, and intratumoural delivery is normal. Synthetic vectors may be used for local/regional delivery without specific targeting ligands or may be equipped with ligands for longer range targeting. Typical ligands are integrin-targeting peptides, but for cancer cells there has been a tendency to use transferrin, anti-transferrin receptor antibodies, or else folate ligands with some degree of success.

In the case of non-viral nucleic acid delivery, nucleic acid constructs may be plasmid DNA (integrating or non-integrating). Alternatively, plasmid mini-circles, cosmids and artificial chromosomes may be used to express nucleic acid encoding a polypeptide of the invention.

In a third aspect, the invention provides an in vitro method of increasing the proliferation of a T cell population, comprising transducing at least one T cell in the population with the vector of the second aspect of the invention. Such a method may be useful in adoptive T cell therapy/adoptive cell transfer (ACT) strategies for the treatment of diseases such as, for example, cancer and chronic viral infections. These strategies are based on expanding target-specific T cells ex vivo and re-infusing the expanded population of T cells into patients. Such methods are known to those skilled in the art and discussed in, for example, WO2006/000830.

In order to utilise Retro in a method of ACT, the following basic steps may be employed: (i) T cells are transduced with a retrovirus encoding a Retro polypeptide of the invention, (ii) the population of T cells is expanded, (iii) the expanded population is then transferred into the patient.

The T cells to be transduced may have been harvested from the patient and may have been selected for target specificity prior to transduction. Alternatively, T cells may be transduced with a nucleic acid encoding a TCR which is specific to a target of interest. The T cells may be CTL or non-CTL cells.

Clinical methods for ACT using endogenous anti-tumour T cells are known in the art. For example Hunder et al. [14] discloses a clinical method for ACT using endogenous anti-tumour CD4⁺ T cells and Yee et al[15] describes a clinical method for ACT using endogenous anti-tumour CD8⁺ T cells.

It is known that endogenous anti-tumour CTLs suffer from functional inactivation and disappearance. Morgan et al. [17] describes a clinical method for ACT using non-specific but functional CTLs transduced with genes encoding TCRs specific for cancer. This is a way of generating tumour-specific CTLs which are capable of functioning in vivo. Thus, non-specific CTLs could be co-transduced with a retrovirus encoding a tumour-specific TCR and a Retro polypeptide of the invention in vitro to produce functional tumor specific CTLs.

ACT can be used to treat solid tumours and can also be used in the treatment of leukemias. The principles of ACT outlined above are also applicable in the case of leukemia treatment.

As mentioned, ACT can also be used to treat chronic viral infection. Diseased caused by HIV, cytomegalovirus (CMV), Epstein-Barr (EBV), Hepatitis B (HBV) and Hepatitis C (HCV) infection may be targeted using ACT. Again the principles of ACT outlined above apply to the treatment of chronic viral infection.

Clinical methods for expansion of anti-HIV CTLs, adoptive transfer and measurement of immunity and HIV levels is described in Tan et al. [33] and Brodie et al. [34].

In addition, Gehring et al. [35] shows the feasibility of using engineered TCRs on CTLs to recognize HBV. This is a way of avoiding functional inactivation/disappearance of CTLs (also described above in relation to anti-tumour CTLs). The method for engineering expression of transgenes in anti-viral CTLs using retroviral transduction described in Gehring et al. is an example of how a nucleic acid encoding a Retro polypeptide of the invention may be introduced into CTLs before adoptive transfer.

For example, to generate human CTL (10⁶/ml) normal donor peripheral blood mononuclear cells (MC) may be cultured with anti-CD3 antibody (OKT3, 50 ng/ml) and IL-2 (600 U/ml) in RPMI (10% FCS) for two days. The CTL may then be transduced with MIGR1 encoding wild-type or mutated Retro (for example E53G human Retro). Transduced CTL can be used after three days [35].

The fourth aspect of the invention provides an isolated T cell containing the expression vector of the third aspect. Such T cells are useful in methods of treating diseases or disorders that would benefit from stimulation of T cell proliferation and/or differentiation, for example a method involving adoptive T cell therapy as outlined above. Thus, the invention also provides a T cell according to the fourth aspect of the invention for use in a method of treating a disease or disorder that would benefit from stimulation of T cell proliferation and/or differentiation. The T cell may be a CTL or a non-CTL T cell.

In a fifth aspect, the invention provides a method of identifying an agent which modulates the binding affinity of the polypeptide of the first aspect of the invention for a target, comprising:
i) contacting the polypeptide or a biologically active fragment thereof with the target and a candidate agent;
ii) measuring the affinity of the polypeptide or biologically active portion thereof for the target; and
iii) comparing the affinity of the polypeptide or biologically active fragment thereof for the target in the presence of the candidate agent with the affinity of the polypeptide or biologically active portion thereof for the target in the absence of the candidate compound,
   wherein an agent which modulates the binding affinity of the polypeptide is identified if the affinity is increased or decreased in the presence of the agent compared to in the absence of the agent.

The skilled person will appreciate that, in order to identify an agent which modulates the binding affinity of Retro for a target, it may not be necessary to contact the agent and the target with a full length sequence of a polypeptide of the invention. The term “biologically active fragment” refers to a portion of the polypeptide which possesses the ability to bind to the target, and thus which may be suitable for use in the method of identifying an agent. “Fragment” refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, at least 250 amino acid residues, at least 275 amino acid residues, at least 300 amino acid residues, at least 325 amino acid residues, at least 350 amino acid residues, at least 375 amino acid residues, or at least 400 amino acid residues) of the amino acid sequence of a polypeptide of the invention.

In some embodiments, the target comprises a nucleic acid sequence. The nucleic acid sequence may comprise a nucleic acid sequence which is similar to or identical to the nucleic acid of a physiological target of Retro. For example, the target may comprise a nucleic acid sequence which is at least 80%, at least 85%, at least 90%, at least 95%, at least 96%, at least 97%, at least 98%, at least 99% or 100% identical to the nucleic acid sequence of a physiological target of Retro. The target may comprise a nucleic acid sequence which is not similar or identical to a physiological target of Retro. Preferably, the nucleic acid sequence is an RNA sequence, for example an mRNA sequence.

The agent may stabilise binding of Retro to the target by binding to Retro at allosteric sites. The allosteric sites may or may not be unique to Retro. In other embodiments, the agent may destabilise binding of Retro to the target by binding to Retro at allosteric sites. Again, the allosteric sites may or may not be unique to Retro.

The method of the fifth aspect of the invention may be adapted to identify agents which interfere with the interaction of a Retro polypeptide with other proteins which associate with Retro in vivo and either augment or reduce the activity of Retro (“associated proteins”). In such an embodiment, a polypeptide of the invention or a biologically active fragment thereof may be contacted with an associated protein in the presence of a candidate agent. A known Retro target, such as an RNA molecule may or may not be present. The affinity of Retro for the protein, the known target, or both may be measured in the presence of the agent and compared the affinity for that entity in the absence of the agent to identify agents which interfere with the interaction of Retro with associated proteins.

The agent may be any molecule which modulates the binding affinity of a polypeptide of the first aspect of the invention for a target. For example, the agent may be a small organic molecule, protein, peptide, antibody or nucleic acid. The method of identifying an agent may involve screening a library of known molecules and/or fragments in order to identify the agent.

The method may comprise the use of a thermostability assay (TSA) and/or Isothermal Titration calorimetry (ITC). TSA is a means of estimating the overall stability of a protein by monitoring the shift in its melting temperature upon changing the buffer conditions or titrating various ligands. The stability of the interaction between protein and ligand is determined by the affinity of the protein for the ligand. The assay is performed by mixing test samples with a fluorophore dye in the wells of a PCR plate. As the protein unfolds with increasing temperature in a thermal cycler, dye molecules bind to exposed core residues and fluoresce, lending to the signal which is measured versus temperature to produce a melt curve. The rate of dye uptake peaks at the melting transition and signals the melting temperature Tₘ; in some cases two or more transitions may occur if the protein contains independently melting domains. Generally, a favourable condition or ligand binding will stabilize a protein and its Tₘ increases—a “positive shift”. Occasionally a condition will induce a “negative shift”, destabilizing the protein and decreasing its Tₘ; some enzyme inhibitors operate in this manner. Further details of TSA can be found in Pantoliano et al. [36]. ITC may be used to confirm and measure binding constants.

Other methods of measuring the affinity of Retro for a target in the presence and absence of an agent will be apparent to the skilled person and fall within the scope of the present invention.

TSA can be developed to measure the thermostability of Retro bound to a target such as RNA and to evaluate the effect of agents such as small molecules on this thermostability. The assay may be validated by the measurement of equilibrium binding constants using ITC to determine the equilibrium binding constant (K_{D}) values and the binding stoichiometries. These methods may utilize multiple RNA oligonucleotides that are known targets of Retro. Several unrelated proteins could be used as a negative control for protein specificity. Should further characterization be required, the method of surface plasmon resonance (SPR) may be used as an alternative or supplemental choice, as it provides for a kinetic measurement of the binding off and on rates.

TSA may be used to identify small molecules and fragments which stabilize Retro:target binding. For fragment screening, multiple concentrations may be screened to identify possible modulators. Several fragment libraries are commercially available and one can be selected to initiate the screening, with evaluation of both positive and negative controls.

Agents which are found to stabilise the binding of Retro to the target may be considered to be activators of Retro function. Agents which are found to destabilise the binding of Retro to the target may be considered to be inhibitors of Retro function. Agents identified using the method of the invention may be modified by computational and/or medicinal chemistry to generate useful enhanced activators or inhibitors of Retro function.

Activators of Retro function may be useful in the treatment, amelioration and/or prophylaxis of a disorder which would benefit from stimulation of T cell proliferation and differentiation, for example CTL proliferation and differentiation, such as cancer. Examples of cancers which the present invention can be used to prevent or treat include solid tumours and leukaemias, including: apudoma, choristoma, branchioma, malignant carcinoid syndrome, carcinoid heart disease, carcinoma (e.g., Walker, basal cell, basosquamous, Brown-Pearce, ductal, Ehrlich tumour, in situ, Krebs 2, Merkel cell, mucinous, non-small cell lung, oat cell, papillary, scirrhous, bronchiolar, bronchogenic, squamous cell, and transitional cell), histiocytic disorders, leukaemia (e.g., B cell, mixed cell, null cell, T cell, T-cell chronic, HTLV-II-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma, chondroblastoma, chondroma, chondrosarcoma, fibroma, fibrosarcoma, giant cell tumours, histiocytoma, lipoma, liposarcoma, mesothelioma, myxoma, myxosarcoma, osteoma, osteosarcoma, Ewing sarcoma, synovioma, adenofibroma, adenolymphoma, carcinosarcoma, chordoma, cranio-pharyngioma, dysgerminoma, hamartoma, mesenchymoma, mesonephroma, myosarcoma, ameloblastoma, cementoma, odontoma, teratoma, thymoma, trophoblastic tumour, adeno-carcinoma, adenoma, cholangioma, cholesteatoma, cylindroma, cystadenocarcinoma, cystadenoma, granulosa cell tumour, gynandroblastoma, hepatoma, hidradenoma, islet cell tumour, Leydig cell tumour, papilloma, Sertoli cell tumour, theca cell tumour, leiomyoma, leiomyosarcoma, myoblastoma, mymoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma, phyllodes, fibrosarcoma, hemangiosarcoma, leimyosarcoma, leukosarcoma, liposarcoma, lymphangiosarcoma, myosarcoma, myxosarcoma, ovarian carcinoma, rhabdomyosarcoma, sarcoma (e.g., Ewing, experimental, Kaposi, and mast cell), neoplasms (e.g., bone, breast, digestive system, colorectal, liver, pancreatic, pituitary, testicular, orbital, head and neck, central nervous system, acoustic, pelvic respiratory tract, and urogenital), neurofibromatosis, and cervical dysplasia, and other conditions in which cells have become immortalised or transformed. In particular, the present invention is useful in the treatment of malignant melanoma, renal carcinoma, prostate cancer, lung cancer, breast cancer and hepatocellular carcinoma. The invention could be used in combination with other treatments, such as chemotherapy, cryotherapy, hyperthermia, radiation therapy, and the like.

Activators of Retro function may also be useful in the treatment, amelioration and/or prophylaxis of chronic viral infections such as those caused by the human immunodeficiency virus (HIV), Epstein-Barr virus, cytomegalovirus and the hepatitis B and C viruses (HBV, HCV).

Increasing the levels of Retro polypeptide in vivo may be useful in treating, ameliorating and/or preventing disorders which would benefit from stimulation of T cell (particularly CTL) proliferation and differentiation. Such disorders as discussed above.

Increasing levels of Retro may be achieved by, for example:
Increasing transcription of the endogenous gene encoding the Retro polypeptide in T cells;
Increasing endogenous Retro mRNA levels in T cells;
Transducing T cells with extra copies of nucleic acid encoding a wild type Retro polypeptide as discussed in relation to the third aspect of the invention.
Transducing T cells with extra copies of nucleic acid encoding a mutant Retro polypeptide as discussed in relation to the third aspect of the invention.

There are numerous methods of increasing levels of a polypeptide such as Retro in vivo. For example, a gene therapy approach may be used in which a nucleic acid encoding a Retro polypeptide of the invention is introduced into a patient's cells in order to boost T cell proliferation and/or differentiation. Alternatively, or additionally, a nucleic acid which encodes a polypeptide other than a Retro polypeptide, for example a transcriptional activator of Retro or a polypeptide which blocks repression of Retro expression may be introduced into a patient's cells. In addition or as an alternative, Retro mRNA processing, transport or translation may be targeted using a method known in the art.

Thus, in one aspect, the invention provides an agent which up-regulates the function and/or expression of the polypeptide of the invention for use in a method of treating or preventing a disease or disorder that would benefit from stimulation of T cell proliferation and/or differentiation, or for use as an adjuvant.

Also provided is a method of treating or preventing treating or preventing a disease or disorder that would benefit from stimulation of T cell proliferation, the method comprising administering an effective amount of an agent which up-regulates the function and/or expression of the polypeptide of the invention to a subject in need thereof.

The agent may be a polypeptide a small organic molecule, protein, peptide, antibody or nucleic acid. In one embodiment, the agent is an oligonucleotide which increases the expression of a polypeptide of the invention. The oligonucleotide may be an oligonucleotide of the invention as defined below. In another embodiment, the agent may affect the function of a polypeptide of the invention by enhancing binding of the polypeptide of the invention to its target.

Reducing the expression and/or activity of Retro may be useful in treating, ameliorating and/or preventing disorders involving unwanted or excessive proliferation and/or differentiation of T cells (such as CTLs) or in clinical situations where immunosuppression is required.

Examples of disorders involving unwanted or excessive proliferation and/or differentiation of T cells such as CTLs include autoimmune diseases and inflammatory diseases such Alopecia Areata, Anklosing Spondylitis, Antiphospholipid Syndrome, Autoimmune Addison's Disease, Autoimmune Hemolytic Anemia, Autoimmune Hepatitis, Autoimmune Inner Ear Disease, Autoimmune Lymphoproliferative Syndrome (ALPS), Autoimmune Thrombocytopenic Purpura (ATP), Behcet's Disease, Bullous Pemphigoid, Cardiomyopathy, Celiac Sprue-Dermatitis, Chronic Fatigue Syndrome Immune, Deficiency Syndrome (CFIDS), Chronic Inflammatory Demyelinating Polyneuropathy, Cicatricial Pemphigoid, Cold Agglutinin Disease, CREST Syndrome, Crohn's Disease, Dego's Disease, Dermatomyositis, Dermatomyositis—Juvenile, Discoid Lupus, Essential Mixed Cryoglobulinemia, Fibromyalgia—Fibromyositis, Grave's Disease, Guillain-Barre, Hashimoto's Thyroiditis, Idiopathic Pulmonary Fibrosis, Idiopathic Thrombocytopenia Purpura (ITP), IgA Nephropathy, Insulin Dependent Diabetes (Type I), Juvenile Arthritis, Lupus, Meniere's Disease, Mixed connective Tissue Disease, Multiple Sclerosis, Myasthenia Gravis, Pemphigus Vulgaris, Pernicious Anemia, Polyarteritis Nodosa, Polychondritis, Polyglancular Syndromes, Polymyalgia Rheumatica, Polymyositis and Dermatomyositis, Primary Agammaglobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjogren's Syndrome, Stiff-Man Syndrome, Takayasu Arteritis, Temporal Arteritis/Giant Cell Arteritis, Ulcerative Colitis, Uveitis, Vasculitis, Vitiligo and Wegener's Granulomatosis. Reducing Retro expression and/or activity may also be useful in treating, ameliorating or preventing symptoms associated with acute and chronic transplant rejection and Graft versus host disease.

Reducing Retro expression and/or activity may be carried out by, for example:
Decreasing target binding, e.g. binding of a Retro polypeptide to a nucleic acid target;
Decreasing transcription of the gene encoding the Retro polypeptide;
Reducing Retro mRNA levels;
Interfering with the interaction of a Retro polypeptide with other proteins that augment the activity of Retro.

Decreasing target binding may be achieved by using a compound identified using a method of the fifth aspect of the invention. For example, compounds which destabilise the interaction of a Retro polypeptide with its target (for example RNA) could be used to interfere with the ability of Retro to bind its target and increase T cell proliferation and/or differentiation.

Decreasing the level of a Retro polypeptide may be achieved by using well-known gene “knock-out,” ribozyme or triple helix methods to decrease expression. In this approach, ribozyme or triple helix molecules are used to modulate the activity, expression or synthesis of the nucleic acid sequence encoding Retro, and thus to ameliorate the symptoms of the disorder. Ribozyme or triple helix molecules may be designed to reduce or inhibit expression of Retro. Techniques for the production and use of such molecules are well known to those of skill in the art.

Endogenous polypeptide expression can also be reduced by inactivating or “knocking out” a Retro gene, or the promoter of such a gene, using targeted homologous recombination (e.g., see Smithies, et al., 1985, Nature 317:230-234; Thomas & Capecchi, 1987, Cell 51:503-512; Thompson et al., 1989, Cell 5:313-321; and Zijlstra et al., 1989, Nature 342:435-438). For example, a Retro gene encoding a non-functional polypeptide (or a completely unrelated DNA sequence) flanked by DNA homologous to the endogenous gene (either the coding regions or regulatory regions of the gene encoding the polypeptide) can be used, with or without a selectable marker and/or a negative selectable marker, to transfect/transduce cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas & Capecchi, 1987 and Thompson, 1989, supra). However this approach can be adapted for use in humans provided the recombinant DNA constructs are directly administered or targeted to the required site in vivo using appropriate viral vectors.

Alternatively, RNA interference (RNAi) may be used to silence or inhibit expression of a Retro gene. mRNA levels may be reduced by, for example, using antisense nucleic acid molecules. For example, expression of Retro may be inhibited by use of antisense nucleic acid molecules. The antisense nucleic acid molecule is capable of hybridising by virtue of some sequence complementarity to a portion of an RNA (preferably mRNA) derived from the nucleic acid sequence of Retro. The antisense nucleic acid may be complementary to a coding and/or non-coding region of a mRNA derived from the DNA encoding a polypeptide of the invention. Such antisense nucleic acids have utility as compounds that inhibit expression, and can be used in the treatment, amelioration, and/or prevention of disorders involving unwanted or excessive CTL proliferation and/or differentiation.

Thus, the present invention provides an oligonucleotide that hybridises to a nucleic acid sequence that encodes the polypeptide of the invention or a nucleic acid sequence that is complementary to a nucleic acid sequence that encodes a polypeptide of the invention.

The invention also provides an oligonucleotide that hybridises to a nucleic acid sequence that controls the expression of a polypeptide of the invention or a nucleic acid sequence that is complementary to a nucleic acid sequence that controls the expression of a polypeptide of the invention. For example, the oligonucleotide may hybridise to a non-coding region such as a promoter or 3′UTR.

The oligonucleotide may be from about 5 nucleotides (nt) to about 1200 nt, about 6 nt to about 500 nt, about 7 nt to about 100 nt, about 10 nt to about 50 nt length or about 20 nt to about 30 nt in length. In some embodiments, the oligonucleotide may be 11, 12, 13, 14, 15, 16, 17, 18, 19, 20, 21, 22, 23, 24, 25, 26, 27, 28 or 29 nt in length. The oligonucleotide may comprise or consist of DNA or RNA. Such oligonucleotides may be useful in methods of treating a disease or disorder that would benefit from stimulation of T cell proliferation and/or differentiation. For example, such an oligonucleotide may stabilise mRNA encoding a polypeptide of the invention leading to increased expression of the polypeptide of the invention.

Oligonucleotides of the invention may be useful in methods of treating or preventing a disease, disorder or condition involving unwanted or excessive proliferation and/or differentiation of T cells. For example, such an oligonucleotide may destabilise or cause the degradation of mRNA encoding a polypeptide of the invention leading to decreased expression of the polypeptide of the invention.

A hybridising nucleic acid molecule or oligonucleotide of the present invention may have a high degree of sequence identity along its length with a nucleic acid molecule encoding a polypeptide of the invention (e.g. at least 50%, at least 75% or at least 90% or 95% sequence identity). As will be appreciated by the skilled person, the higher the sequence identity a given single stranded nucleic acid molecule has with another nucleic acid molecule, the greater the likelihood that it will hybridise to a nucleic acid molecule which is complementary to that other nucleic acid molecule under appropriate conditions. The “percent identity” discussed above in relation to the first aspect applies equally to this aspect. Hybridisation may take place under the following conditions: 400 mM NaCl, 40 mM PIPES pH 6.4, 1 mM EDTA, 50° C. or 70° C. hybridisation for 12-16 hours; followed by washing

The invention also provides an agent which down-regulates the function and/or expression of the polypeptide of the invention for use in a method of treating a disease, disorder or condition involving unwanted or excessive proliferation and/or differentiation of T cells.

Also provided is a method of treating or preventing a disease, disorder or condition involving unwanted or excessive proliferation and/or differentiation of T cells, the method comprising administering an effective amount of an agent which down-regulates the function and/or expression of the polypeptide of the invention to a subject in need thereof.

The agent may be a polypeptide a small organic molecule, protein, peptide, antibody or nucleic acid. In one embodiment, the agent is an oligonucleotide of the invention as defined above.

Agents of the invention may be provided in the form of a pharmaceutically acceptable composition. Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Where the formulation is a liquid it may be, for example, a physiologic salt solution containing non-phosphate buffer at pH 6.8-7.6, or a lyophilised powder.

Other suitable routes of administration include intravenous, subcutaneous, intradermal, intraperitoneal and intramuscular administration. Liquid formulations may be utilised after reconstitution from powder formulations.

For intravenous injection, or injection at the site of affliction, the active ingredient will be in the form of a parentally acceptable aqueous solution which is pyrogen-free, has suitable pH, is isotonic and maintains stability. Those of relevant skill in the art are well able to prepare suitable solutions using, for example, isotonic vehicles such as sodium chloride injection, Ringer's Injection or Lactated Ringer's Injection. Preservatives, stabilisers, buffers, antioxidants and/or other additives may be included, as required.

The composition may be administered in a localised manner to, for example, a tumour site or other desired site or may be delivered in a manner in which it targets tumour or other cells.

The compositions are preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. The compositions of the invention are particularly relevant to the treatment of cancer, and in the prevention of the recurrence of such conditions after initial treatment or surgery.

Examples of the techniques and protocols mentioned above can be found in Remington's Pharmaceutical Sciences [40]. A composition may be administered alone or in combination with other treatments, either simultaneously or sequentially dependent upon the condition to be treated. The compositions of the present invention may be generated wholly or partly by chemical synthesis. The composition can be readily prepared according to well-established, standard liquid or, preferably, solid-phase peptide synthesis methods, general descriptions of which are broadly available (see, for example, in Solid Phase Peptide Synthesis, 2^{nd} edition [41], in The Practice of Peptide Synthesis [42] and Applied Biosystems 430 A Users Manual, ABI Inc., or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

In a further aspect, the invention provides a method of identifying agents that modulate (i.e., upregulate or downregulate) the expression and or activity of a polypeptide of the invention (“a Retro polypeptide”).

Such agents may be identified by contacting T cells, preferably CTLs expressing the Retro gene with a candidate agent or a control (e.g., phosphate buffered saline (PBS)) and determining the expression of the Retro gene or mRNA encoding a polypeptide of the invention (a “Retro polypeptide”). The level of expression of the Retro gene or mRNA encoding a Retro polypeptide in the presence of the candidate agent is compared to the level of expression of the Retro gene or mRNA encoding a Retro polypeptide in the absence of the candidate agent (e.g., in the presence of a control). The candidate agent can then be identified as a modulator of the expression of Retro. When expression of the Retro gene or mRNA encoding a Retro polypeptide is significantly less in the presence of the candidate agent than in its absence, the candidate agent is identified as an inhibitor of the expression of the Retro gene or mRNA encoding a Retro polypeptide. The level of expression of a Retro polypeptide or the mRNA that encodes it can be determined by methods known to those of skill in the art. For example, mRNA expression can be assessed by Northern blot analysis or RT-PCR, and protein levels can be assessed by western blot analysis.

The invention also provides a kit comprising a nucleic acid probe capable of hybridising to nucleic acid encoding a polypeptide of the invention. In a further aspect there is provided a kit comprising in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding a polypeptide of the invention, such as by polymerase chain reaction (see e.g., Innis et al., 1990, PCR Protocols, Academic Press, Inc., San Diego, Calif.), ligase chain reaction (see EP 320,308) use of Q13 replicase, cyclic probe reaction, or other methods known in the art.

Preferred features of each aspect of the invention are as for each of the other aspects mutatis mutandis. The prior art documents mentioned herein are incorporated to the fullest extent permitted by law.

### EXAMPLES

### Example 1

### Identification of a Positive-Regulator of Tumour Immunity

Many targets that show animal model efficacy not only control immunosuppression of anti-tumour CTLs but also immunosuppression of anti-viral CTL responses (e.g. PD-1, IL-2, OX-40). A model system of immunosuppression during chronic LCMV infection was used to identify new pathways for the control of T cell immunosuppression. An unbiased forward genetic approach to identify mouse mutants with CTLs that are resistant to immuno-suppression was used. Using ethyl-nitrosourea (ENU) mutagenesis random mutations in the sperm of C57BL/6J (Charles River sub-strain) male mice (GO) were generated. GO mice were bred to C57BL/6J wild-type females to obtain a G1 generation mice derived from an ENU-mutated sperm with a unique spectrum of point mutations (about 2,500 heterozygous mutations per G1 [27], [28]). Two subsequent crosses brought mutations to homozygosity in G3 animals. Infection of mice with the clone 13 variant of LCMV (LCMV C13) results in chronic infection because of physical deletion of protective CTLs specific for the np396 epitope. 403 G3 mice were screened for mutants that were resistant to the deletion of np396 specific CTLs after LCMV C13 infection.

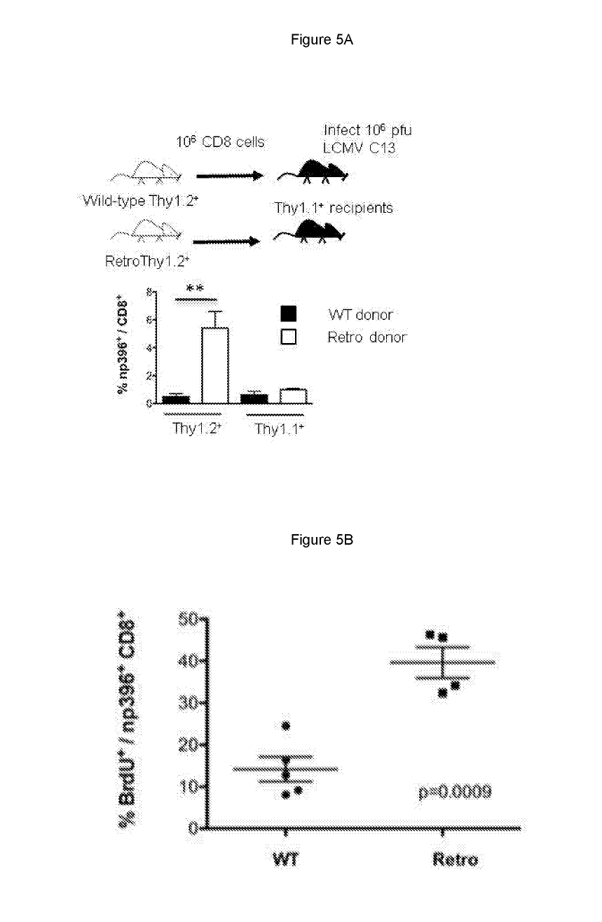

G3 mice were identified from one G1×G2 breeding pair that had a >8-fold increase in the number of np396-specific CTLs, increased cytolytic activity (based on surface CD107α expression) and the down-regulation of PD-1 expression. G3 with this phenotype were called Retro mice. The Retro phenotype was inherited as a semi-dominant trait and homozygous mutant Retro mice were generated. Analysis of Retro homozygous mutant mice revealed that they had about 20-fold greater number of np396 specific CTLs compared to wild-type (FIG. 4) and a corresponding 10⁴-fold decrease in the titer of LCMV C13 in the spleen (data not shown). This increased immunity to LCMVC13 came at the cost of increased immunopathology because there was a substantial increase in mortality of Retro homozygous mice (data not shown). Naïve Retro mutant mice harbour increased levels of activated T cells and exhibit signs of autoimmunity late in life (data not shown).

### Example 2

### Increase in Np396-Specific CTL is Cell-Autonomous to CD8 T Cells

1×10e7 CD8 cells were isolated by CD8a (Ly2) beads (Miltenyi Biotec) from donor Retro Thy1.2 and WT Thy1.2 mouse spleens and intravenously injected into 3-4 WT B6 recipients Thy 1.1 mice of 6-8 weeks old with sterile PBS used as a control. After 24 hour recovery from the injection, recipient mice were infected with 1×10e6 pfu LCMV C13. On day 8 post infection, spleens were taken from the mice and standard staining with CD8 antibody, LCMV NP396 and GP33 tetramers were performed for measurement of CD8NP396 and CD8GP33 antigen specific cells using FACS on a CyAn ADP (Dako).

This experiment showed that the increase in the number of np 396-specific CTLs after LCMV C13 infection was a trait that was cell-autonomous to CD8 T cells (FIG. 5A).

### Example 3

### Retro Homozygous Mutant CTL Proliferated More than Wild-Type after LCMV C13 Infection

4-5 Retro homozygous mice fed on BrdU containing H₂O were intravenously infected with 1×10e6 pfu LCMV C13 using WT B6 as controls. On day 8 post infection, splenocytes were isolated and stained with anti-CD8 antibody and LCMV NP396 and GP33 tetramers following standard BrdU and AnnexinV stainings according to the manufacture's protocol (eBioscience). The frequencies of BrdU/Annexin V positive in antigen specific CD8 T cells were measured by standard FACS on a CyAn ADP (Dako) using proper gating.

This study revealed that Retro homozygous mutant CTLs proliferated more than wild-type after LCMV C13 infection (FIG. 5B). No difference in the percentage of cells undergoing apoptosis as indicated by annexin V staining was observed.

### Example 4

### The Identification of the Retro Mutation

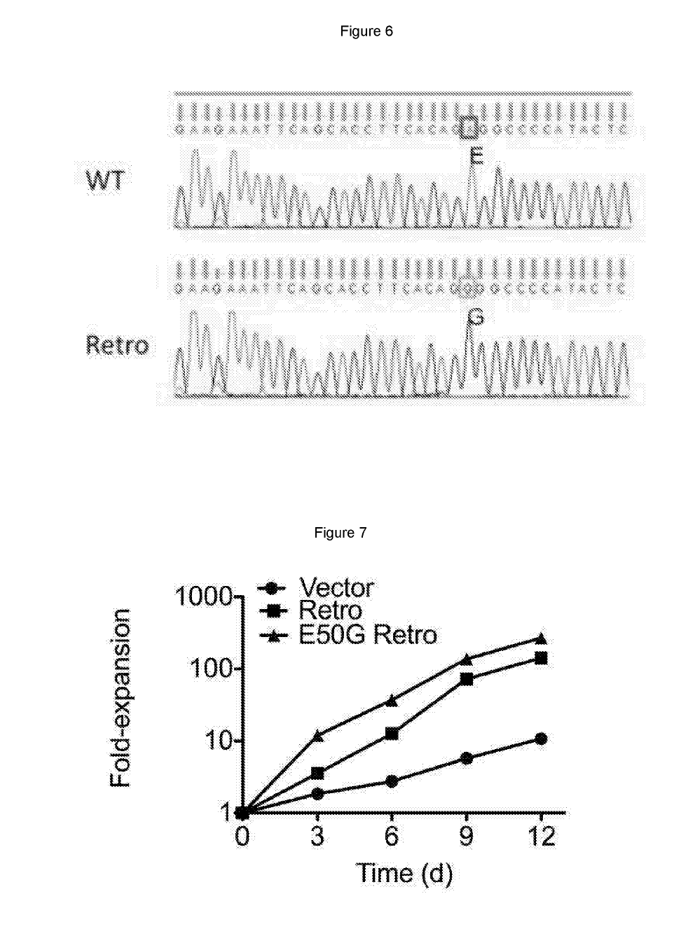

Genomic DNA from Retro homozygous mutant mice was subjected to whole exome next generation sequencing. Comparison with the wild-type C57BL/6 reference exome sequence (sub-strain: C57BL/6J) identified 8 homozygous single nucleotide polymorphisms (SNP) that would give synonymous amino acid sequencing revealed that 4/8 of the Retro-associated SNP were also present in the genome of wild-type C57BL/6 sub-strain (Charles River) used for our ENU mutagenesis. The 4 ENU-generated SNP were then analysed in the progeny of mice to see which one segregated with the Retro phenotype. Only one Retro mutant allele was found to be homozygous in every mouse exhibiting the Retro phenotype (>4-fold increase in level of np396+CD8+ cells on day 8 of C13 infection). This homozygous SNP was in the gene BC055111 on chromosome 4 resulting in a glutamate (E) to glycine (G) change at amino acid 50 (E50G) (FIG. 6).

### Example 5

### Validation of BC055111 as the Retro Gene

The in vivo findings discussed above show that Retro mutant mice harbouring the BC055111 E50G mutation have increased levels of CTLs due to increased proliferation. To validate the E50G mutation in BC055111 as the causative mutation for the Retro-phenotype, wild-type or E50G mutant BC055111 were overexpressed as open reading frames (ORF) in the mouse CTLL-2 T cell line [29] by MIGR1 retrovirus transduction [30]. Over-expression of wild-type BC055111 resulted in a 14-fold increase in the expansion of CTLL-2 cells compared to empty vector controls (FIG. 7). CTLL-2 cells transduced with E50G mutant BC055111 expanded about 3-times more than cells with wild-type BC055111. Real-time PCR revealed that the level of BC055111 mRNA was the same in CTLL-2 cells over-expressing E50G mutant versus wild-type ORF (data not shown).

Therefore, wild-type Retro promotes the proliferation of CTLs and the E50G mutation is a gain-of-function phenotype. It was concluded that E50G mutation in BC055111 is the causative mutation of the Retro phenotype and so the BC055111 gene will be referred to as the Retro gene.

### Example 6

### Increased Retro Gene Expression in Retro Mutant CTLs

The expression of Retro mRNA in CD8 T cells was examined after activation by anti-CD3 and anti-CD28 antibodies. 24 well plates were coated with monoclonal anti-CD3 antibody 1 μg/ml) and (2 μg/ml) monoclonal anti-CD28 antibody at 4° C. overnight. Primary CD8 T cells (magnetic bead sorted) from both wild-type and Retro mutant mice (1.5×10⁶) were cultured in RPMI-10% FCS on antibody coated plates and IL2 (5 ng/ml). The relative Retro mRNA level determined by real-time PCR is normalized by the level in naïve CD8 T cells and the GAPDH internal control.

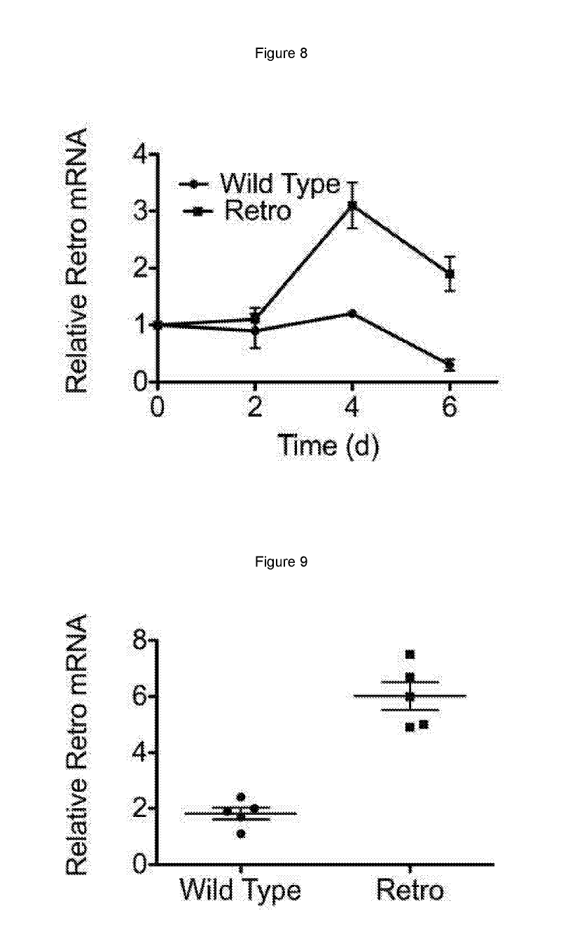

A modest (1.2-fold) up-regulation of BC055111 mRNA on day 4 of culture was observed, which then diminished to the naïve level by day 6. However, when CD8 T cells from Retro mutant mice were examined, a far greater up-regulation (3-fold) of BC055111 on both day 4 and day 6 (2-fold) over the naïve level was observed (FIG. 8). The in vivo relevance of this observation was confirmed when a 6-fold up-regulation in BC055111 mRNA in anti-LCMV CTLs in Retro mutant compared to a 2-fold in wild-type mice was also observed (FIG. 9). Data shown is for magnetic bead sorted CD8 T cells on day 8 of infection with LCMV C13 (10⁶ pfu/i.v.). The relative Retro mRNA level determined by real-time PCR is normalized by the level in naïve CD8 T cells and the GAPDH internal control. Mean values from 6 mice are shown.

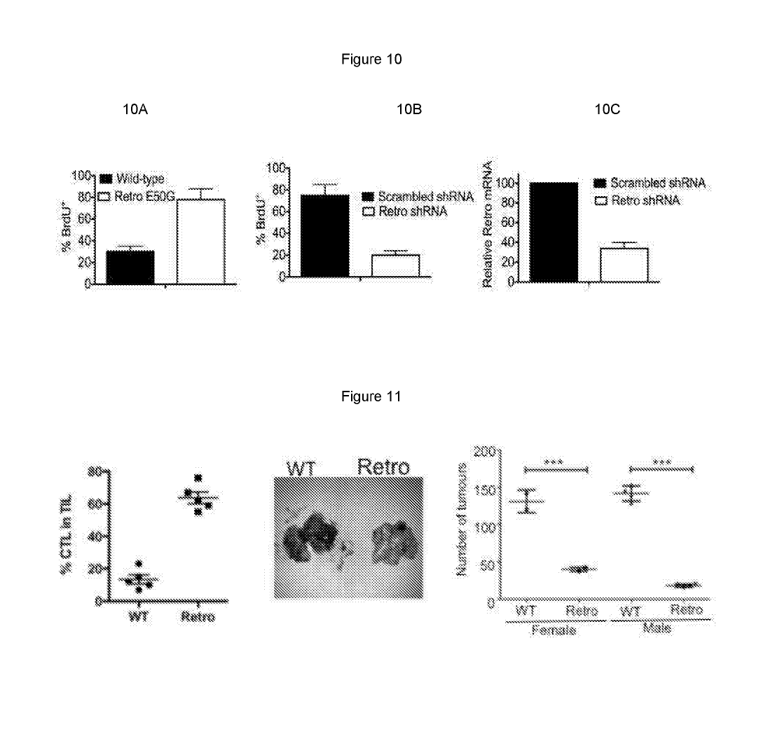

As observed in vivo (FIG. 5), after activation (by TCR/CD28 stimulation), CD8 T cells from Retro mutant mice were hyper-proliferative (as evidenced by BrdU incorporation) (FIG. 10A). Briefly, CD8 T cells from wild-type and Retro mutant mice were cultured on anti-CD3 and anti-CD28 antibodies (as in FIG. 10B) for 3 days, then pulsed with BrdU and on day 4 the % that stained positive with anti-BrdU⁺ antibody (PE-secondary antibody) then determined. Mean values from 4 wells are shown.

To determine if the increased level of BC055111 mRNA contributed to the hyper-proliferation of Retro CD8 T cells, BC055111 mRNA was knocked down in Retro mutant CTLs. CD8 T cells from Retro mutant mice were cultured on anti-CD3 and anti-CD28 antibodies for 2 days then transduced with lentivirus (GIPZ vector, Open Biosystems) (M01=30:1) encoding shRNA encoding either scrambled shRNA or shRNA specific for BC055111 (according to manufacturer's protocol). On day 3 cells were pulsed with BrdU and on d4 the % of GFP+ cells (5-20%) that stained positive with anti-BrdU+ antibody (PE-secondary antibody) was determined. Mean values from 4 wells are shown in FIG. 10B.

CD8 T cells from Retro mutant mice were cultured on anti-CD3 and anti-CD28 antibodies for 2d then transduced with lentivirus then on day 4 GFP⁺ cells purified by FACS. The relative Retro mRNA level was determined by real-time PCR compared to GAPDH internal control and expressed as % of the level in GFP⁺ cells transduced with scrambled shRNA. Mean values from 4 wells are shown in FIG. 10C.

The proliferation of Retro mutant CTLs transduced by Retro shRNA was reduced by about 3-fold (FIG. 10B) when the level of BC055111 mRNA in day 4 Retro mutant CTLs was knocked down by 70% (FIG. 10C). It was concluded that the increased expression of BC055111 mRNA contributes to the Retro phenotype of CTL hyper-proliferation.

### Example 7

### Retro Mutant Mice have Increased CTL Immunity to Melanoma

Some negative checkpoints that control CTL-immunity to chronic viral infection also control CTL-immunity to cancer, the best known being the PD-1/PD-L1 axis [31, 32 and 11]. Therefore CTL-immunity to malignant melanoma was examined in the B16 melanoma transplantation model. Mice were injected (i.v.) with B16-F10 melanoma cells (C57BL/6 origin) (3×10⁵) then after 5 weeks melanomas analyzed in the lungs. Tumours were excised and digested and purified on a ficol gradient and the % of CD3⁺ CD8⁺ (CTL) determined.

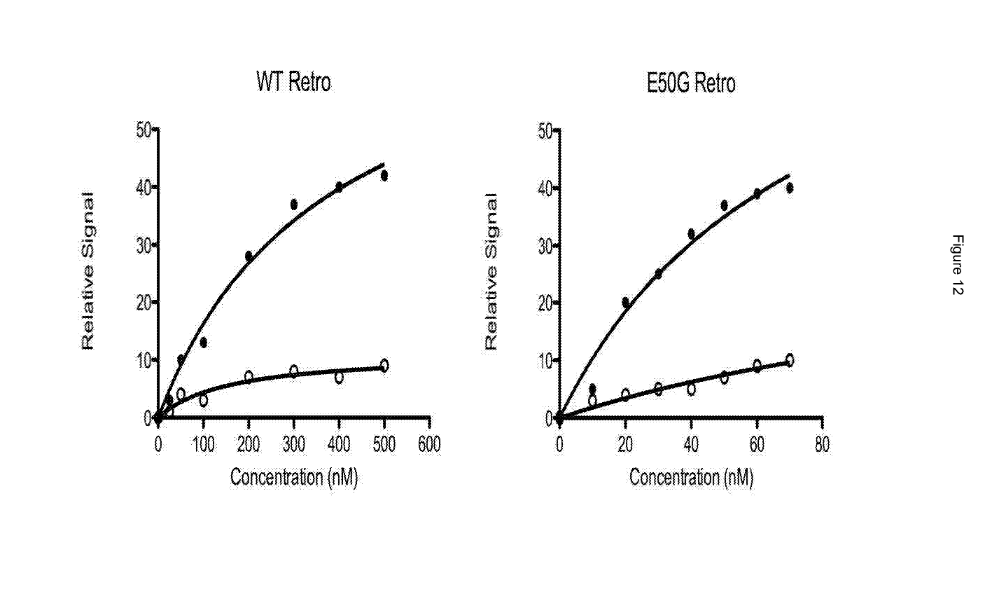

FIG. 11 (left panel) shows that Retro homozygous mutant mice generated about 6-times more CTLs in TIL compared to wild-type. The middle panel of FIG. 11 is a picture of lungs showing melanoma tumour foci in black. The right panel of FIG. 11 shows the number of melanoma tumour foci in female or male mice in WT (n=6) or Retro (n=6) mice. There was about 5-fold fewer tumours in the lungs of Retro mice compared to wild-type. ***p<0.001. This experiment is representative of 3 others. Although it is difficult to compare with other independent studies the magnitude of increase in the level of CTLs in TIL from B16-BL6 melanoma compares favourably with the 3-fold increase induced by GVAX vaccination followed by PD-1 and CTLA-4 antibody combined blockage [11].

### Example 8

### Retro is an RNA-Binding Protein

Wild-type or E50G BC055111 ORFs (constructed by GeneArt, Life Technologies, Invitrogen) were cloned into the pEX6 vector and GSTRetro produced in E. coli then purified on GST beads to >90% purity. GST-Retro protein (74 kD, 150 ng) was incubated with biotin-labeled 5′-UUUAUUUAUUAUU-3′ (over a range of concentrations) as in Barreau, 2005 #308 [23]. Then the binding of labeled oligoribonucleotide to Retro was measured after filtration through nitrocellulose followed by washing using a slot blotter. RNA bound to Retro on filters was visualized by probing with streptavidin-HRP and developed with ECL. The relative signal was determined by densitometry on slot signals. Relative signal was determined from the binding of biotin labeled oligoribonucleotide alone (filled circles in FIG. 12) or in the presence of a 100-fold molar excess of un-labeled oligoribonucleaotide (open circle in FIG. 12).

These assays revealed that affinity (Kd) of binding between recombinant mouse Retro and an oligoribonucleotide containing an ARE motif was about 153 nM (FIG. 12). It was also found that the E50G mutant version of Retro bound to the ARE oligoribonucleotide with a higher affinity (Kd=34 nM). It was concluded that the E50G mutation increases the affinity of Retro for this particular RNA species. The affinity of Retro binding to the ARE motif is within the affinity range of bone fide RNA-BPs with specificity for mRNA

### Example 9

### Expression and Purification of a Retro Polypeptide

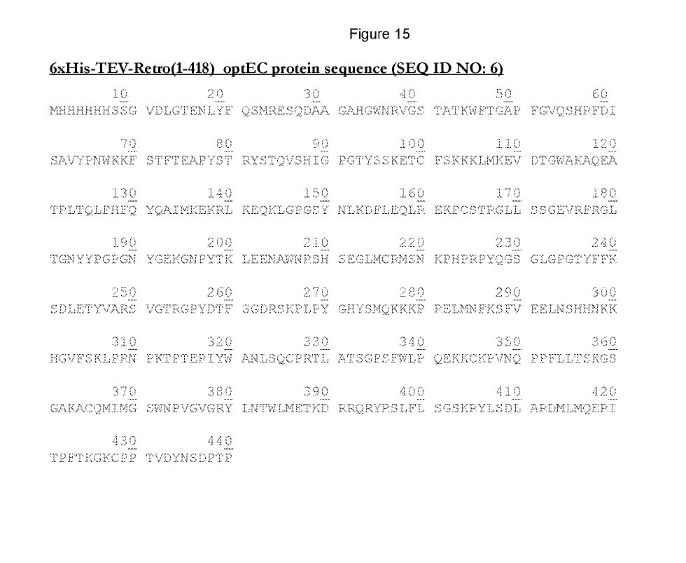

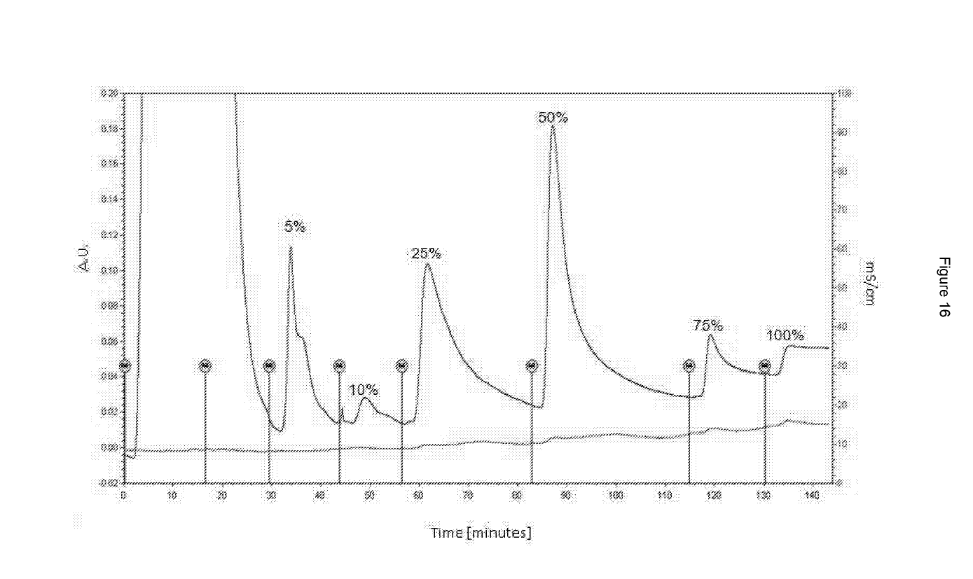

Expression: A vector containing the 6×His-TEV-retro(1-418) optEC (the DNA sequence of which (SEQ ID NO: 3) is shown in FIG. 13) was transformed into E. coli BL21 (DE3) cells and plated on an L.B. agar plate. A plasmid map of the vector is shown in FIG. 14. The protein sequence of 6×His-TEV-retro(1-418) optEC is shown in FIG. 15. A single colony was selected and inoculated into a 10 mL culture of T.B. media. The culture was incubated at 37° C. overnight. A 1 mL aliquot of the overnight culture was inoculated into a fresh 1 liter flask of T.B. media (×5). The cultures were grown to OD600=0.6 and induced with 1 mM IPTG. The cultures were incubated for 3 hours at 37° C. Cells were harvested by centrifugation at 6,000 RPM for 20 minutes. A wet pellet of 10.3 grams was recovered (Lot#: X142-023) and stored at −80° C.

Cell Lysis and Clarification: Cell pellet X142-023 was removed from the freezer and thawed on ice. The cell pellet was suspended in 200 mL's of lysis buffer and disrupted by passing the homogenous solution through a microfluidizer. The lysate was clarified by centrifugation at 10,000 RPM for 30 minutes and filtered at 0.45 μm.

Ni Affinity Chromatography: A 5 mL Ni-NTA column was equilibrated with 50 mL's of PBS buffer. The clarified lysate was passed over the column at 2 mL's per minute. The column was washed to baseline and eluted using a series of step gradients (5%, 10%, 25%, 50%, 75%, 100%). A chromatogram is presented in FIG. 16.

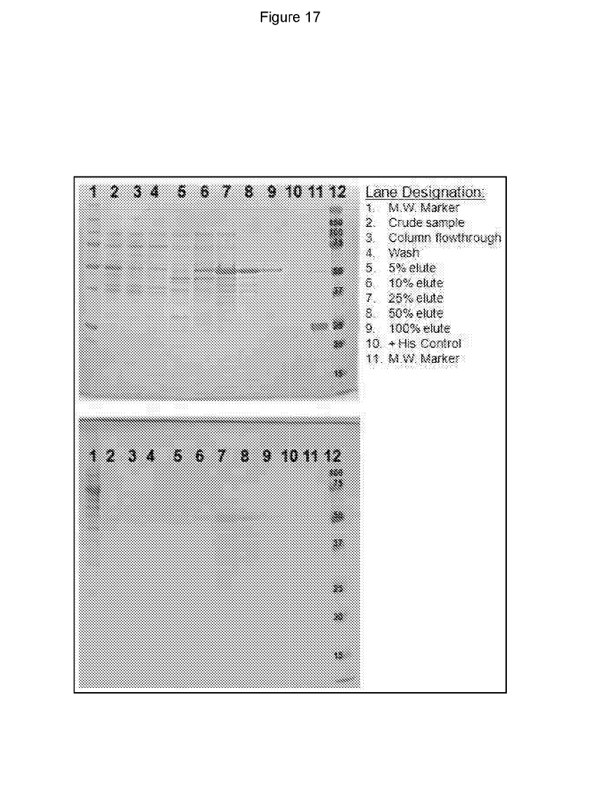

An analysis of the pre-column and elution fractions is presented in FIG. 17. The sample that eluted at 250 mM imidazole was concentrated to 1 mg/mL and used for testing refolding conditions.

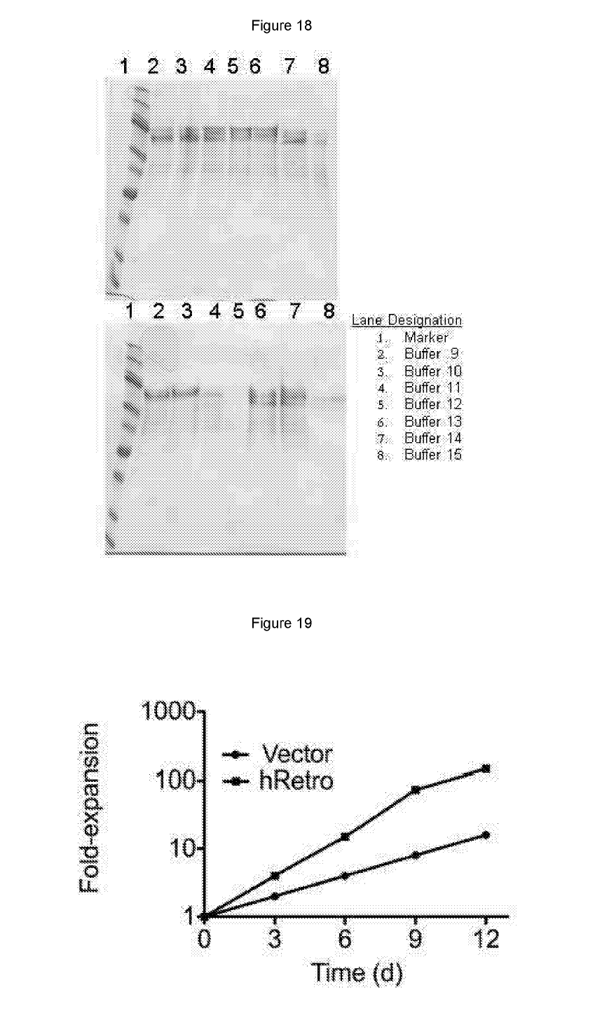

Refolding of 6×His-TEV-Retro(1-418) optEC: A 1 mL sample of the Ni-NTA purified material was refolded by two methods. In one trial the sample was directly dialyzed against 1 liter of buffer with no urea. In trial number two the sample was refolded by slow dilution. The 1 mL sample was placed into 100 mL of unfolding buffer and diluted to 2,100 mL over 16 hours. The dialyzed samples were centrifuged at 13,000 RPM for 20 minutes. After centrifugation a white pellet of precipitated material was observed. A Bradford assay of the soluble fraction showed no measurable protein concentration. In a second refolding trial, the Retro protein sample was dialyzed against a buffer containing 500 mM Sodium chloride, all other buffer components were held the same as described above. All material was found in the precipitated form after centrifugation. In a third refolding trial the Retro protein was dialyzed against a series of 15 complex buffers in a propriety refolding screen. An SDS-PAGE analysis of the refolded, soluble fractions of each condition showed that two buffers resulted in stable material that contained only one band in a Coomassie gel (FIG. 18).

### Example 10

### Overexpression of hRetro Increases Expansion of Jurkat T Cells

Flag-C1orf177 was commercially synthesized into the pcDNA3.1(+) vector (GeneART technologies, Invitrogen). Flag-C1orf177 coding sequences were excised from pcDNA3.1(+)-C1orf177 by digestion with Xhol and cloned into the MIGR1 vector. Constructs were verified by automated DNA sequencing for correct orientation. MIGR1-C1orf177 was transiently transfected into the Phoenix packaging line using calcium phosphate method (CAPHOS-1KT, Sigma) in a 100 mm TC plate. Twenty four hours following transfection, media was removed and the cells washed gently with PBS, and fresh media re-applied. Cells were transferred to a 32° C., 5% CO₂ incubator and left overnight. The following morning polybrene (5 ug/ml) was added to the virus containing media and gently agitated. Virus containing media was removed and filtered through a 0.45 uM filter to avoid cell carry over. Fresh media was applied to the Phoenix cells. Virus containing media was added to 5×105/ml Jurkat cells and centrifuged at 2250 rpm for 90 mins at 37° C. and left at 32° C. 5% in a TC incubator for eight hour incubation. Following incubation, the Jurkat media was removed, and replaced with fresh virus containing media, spun and kept at 32° C. overnight. The cycle of viral infection was continued for three days dependent on the condition of the phoenix cells. After this time transduction was measured as % GFP⁺ in Jurkat cells (typically 5-20%).

The number of transduced cells was determined (GFP⁺) on day 0, then every 3 days until day 12. The fold expansion was determined by dividing through by the starting number on day 0. FIG. 19 shows that over-expression of wild-type human Retro increases the expansion of Jurkat cells. Data shown is the mean±sem (n=4 wells).

### Example 11

### Compound Screen

A Thermal Stability Assay (TSA) specifically designed for Retro was performed as follows. Refolded protein was desalted in 2.5-mL aliquots using PD-10 spin columns (Sephadex G-25, GE 17-0851). At this point the protein was approximately 5 μM in 50 mM Tris-HCl pH 8.5, 10 mM sodium chloride, 0.4 mM potassium chloride, 750 mM guanidinium chloride and 0.05% PEG 3350. The desalted aliquots were pooled together and a portion was used to form the Retro/RNA complex. This involved the addition of 25 μM of the oligonucleotide 5′-UUUAUUUAUUAUU-3′ (Boston Open Labs) and incubation with gentle rocking overnight at 4° C.

For a 96-well assay, 2.5 mL of protein was mixed via aspiration with 5 μL of CPM dye in DMSO to yield a dye concentration of 0.2 mM. In a clear 96-well PCR plate, 24 μL of the protein plus dye was placed in every well. Then, using a 12-channel pipet and gentle mixing, 1 μL of test compound was added per well. The well contents were visually inspected and remixed where necessary since some of the compounds showed dissolution. In total, 352 compounds were tested using Fragment Library 1 from Zenobia Therapeutics. The compounds were stored frozen in four 96-well plates at 200 mM in DMSO and for the most part the plate array was replicated onto the assay plates. At assay time, each well contained 8 mM compound and 4% DMSO. Two wells per plate were used for control samples (DMSO and buffer), so 94 compounds were tested on the first three plates and the hold-outs were tested on the fourth plate (which had a total of 70 test compounds).

The entire assay was repeated with buffer only (no protein) to test the background signal attributed to each compound. The Retro/RNA complex was tested against all 352 compounds but the Retro protein alone (no RNA) was tested only against the first 94 compounds from Fragment Library Plate 1.

Compounds were ranked in order of their thermal shift values relative to the baseline value of 38° C. Multiple compounds that positively stabilize the Retro-RNA complex were identified.

### Example 12

### Increased Development of Memory CD8 T Cells in Retro Mutant Mice

Infection of mice with the WE strain of LCMV gives an acute infection that results in a robust primary CD8 T cell response, long-term immunological memory and viral clearance. The development of memory T cells benefits vaccination. Thus infection of mice with the WE strain of LCMV can effectively vaccinate against subsequent challenge with a strain of LCMV that would give chronic infection in un-vaccinated mice (e.g. LCMV C13 strain). In this Example, LCMV WE infection was used as a measure of the effect of the Retro mutation on memory T cell development and therefore the potential to increase the efficacy of vaccination.

CB57 BL/6 wild type and Retro homozygous mutant mice were infected with LCMV WE (200 pfu i.p.) Flow-cytometry after staining with MHC-tetramers containing either the gp33 or np396 peptide antigens of LCMV (Lymphocytic Choriomenigitis virus) and anti-CD8 antibody was used to measure the percentage of CD8+ T cells specific for gp33 or np396 of total T cells in peripheral blood leucocytes over time.

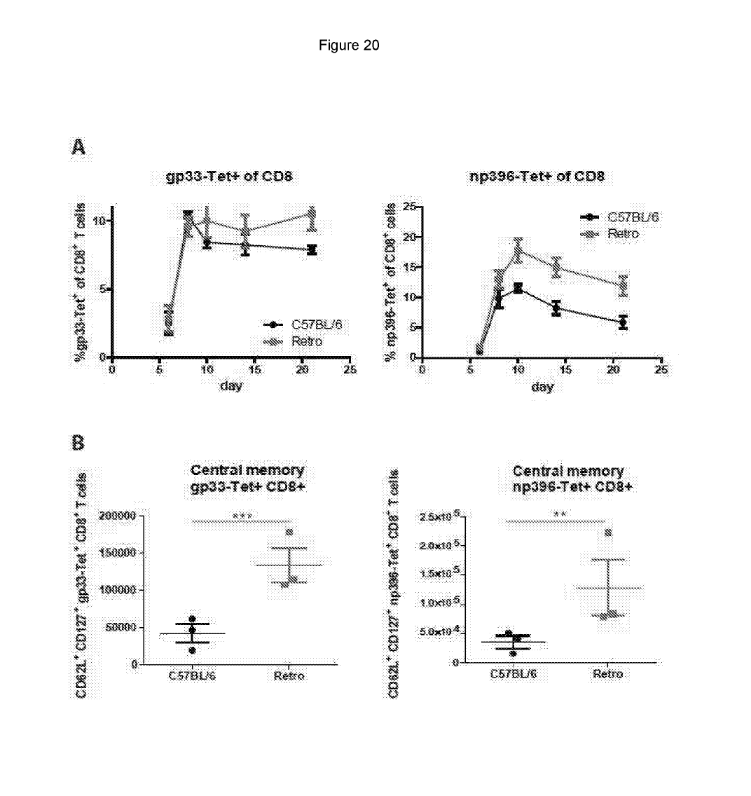

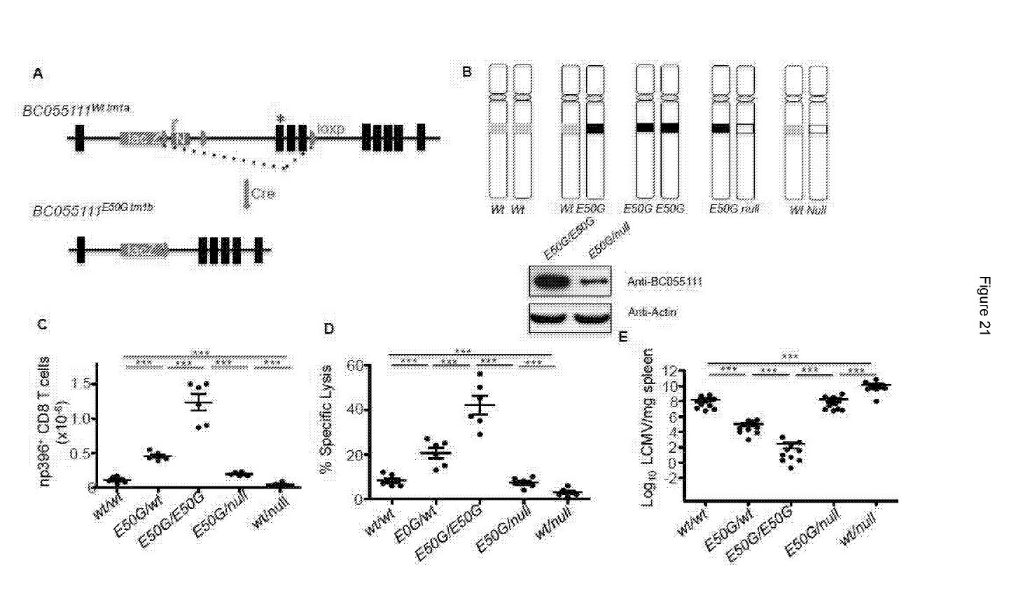

The results are shown in FIG. 20A (n=4 mice). Clonal bursts of CD8 T cells specific for both the gp33 and np396 antigens can be seen from day 8 post infection. In mice having the Retro mutation, the percentage of gp33 and np396-specific CD8 T cells out of total T cells was significantly increased relative to the percentage observed in the wild type mice.

Subsequently, the number of gp33 and np396-specific central memory CD8 T cells was measured in the spleen after 21 days. The results are shown in FIG. 20B (n=4 mice). It can be seen that the Retro mutant mice had significantly more central memory CD8 T cells than wild type mice 21 days post-infection.

### Example 13

### In Vivo Validation of the Causative Retro Mutation

In order to examine whether BC055111 (the mouse Retro gene) directly controls CTL immunity to LCMV, mice with a targeted BC0551111 null allele were generated. Mice harbouring the BC055111t^{m1a} allele were obtained from the KOMP (Knock-out mouse project) repository then bred with transgenic mice constitutively expressing Cre recombinase under the control or the Ella promotor [37] to delete exons 2-4 after recombination between loxp sites. The position of the E50G mutation in exon 2 is indicated in FIG. 21A by the asterisk. Inter-crossing generated mice harbouring different combinations of BC055111 alleles (illustrated in FIG. 21B). The generation of the BC055111t^{m1b} null allele was verified by PCR (using primers that spanned the exons deleted by Cre recombination) followed by Western blot (anti-BC055111 antibody from Santa Cruz Biotech, 1/200 dilution) which showed a 50% reduction for BC055111 protein in BC055111^{E50G/null} compared to BC055111^{E50G/E50G} mice (FIG. 21B).

Mice harbouring the different allele combinations set out in FIG. 21B were infected with LCMV C13. On day 8 the level of np396⁺ CD8 T cells in the spleen was determined by tetramer staining and flow-cytometry. The results are shown in FIG. 21C which indicates that the E50G/E50G allele combination produced the highest number of np396⁺ CD8 T cells and the wt/null combination produced the lowest number of np396⁺ CD8 T cells In addition, it can be seen that the E50G/null mutation produced fewer np396⁺ CD8 T cells than the E50G/wt allele combination.

The cytotoxic activity (E/T=10:1, np 396-pulsed EL4 targets) of the T cells of each mouse type was determined using standard CTL assays [36]. A pattern of results similar to that shown in FIG. 21C was observed, with the E50G/E50G combination giving rise to the highest cytotoxic activity, the wt/null combination having the lowest cytotoxic activity and the E50G/null combination producing lower cytotoxic activity than the E50G/wt combination (see FIG. 21D).

Finally, the titre of LCMV was determined for each allele combination using a PCR assay [39] (n=6-10 mice. ***p<0.001). The results are shown in FIG. 21E. Mice with the E50G/E50G allele combination achieved the lowest viral titre and mice with the wt/null combination had the highest viral titre. Mice with the E50G/null allele combination had a higher viral titre than mice with the E50G/wt combination.

In summary, it has been determined that in BC055111^{E50G/null} mice, the null allele negates the Retro gain-of function phenotype in trans, indicating that the E50G mutation is the culpable mutation for the Retro phenotype. In addition, BC055111^{Wt/null} mice had decreased anti-LCMV CTL immunity compared to BC055111^{Wt/Wt} mice indicating that BC055111 is a positive regulator of CTL expansion. This also indicates that ablation of Retro expression (in this case by gene knock-out) can reduce the expansion of T lymphocytes in vivo.

### REFERENCES

1. Pardoll, D. T cells take aim at cancer. Proc Natl Acad Sci USA 2002; 99: 15840-2.
2. Schwartz, R H. Acquisition of immunologic self-tolerance. Cell 1989; 57: 1073-81.
3. Atkins, M B, Kunkel, L, Sznol, M, and Rosenberg, S A. High-dose recombinant interleukin-2 therapy in patients with metastatic melanoma: long-term survival update. Cancer J Sci Am2000; 6 Suppl 1: S11-4.
4. Kirkwood, J M, Manola, J, Ibrahim, J, Sondak, V, Ernstoff, M S, and Rao, U. A pooled analysis of eastern cooperative oncology group and intergroup trials of adjuvant high-dose interferon for melanoma. Clin Cancer Res 2004; 10: 1670-7.
5. Asadullah, K, Sterry, W, and Volk, H D. Interleukin-10 therapy—review of a new approach. Pharmacol Rev 2003; 55: 241-69.
6. Diehl, L, den Boer, A T, Schoenberger, S P, van der Voort, E I, Schumacher, T N, Melief, C J, Offringa, R, and Toes, R E. CD40 activation in vivo overcomes peptide-induced peripheral cytotoxic T-lymphocyte tolerance and augments anti-tumor vaccine efficacy. Nat Med 1999; 5: 774-9.
7. French, R R, Chan, H T, Tutt, A L, and Glennie, M J. CD40 antibody evokes a cytotoxic T-cell response that eradicates lymphoma and bypasses T-cell help. Nat Med 1999; 5: 548-53.
8. Sotomayor, E M, Borrello, I, Tubb, E, Rattis, F M, Bien, H, Lu, Z, Fein, S, Schoenberger, S, and Levitsky, H I. Conversion of tumor-specific CD4+ T-cell tolerance to T-cell priming through in vivo ligation of CD40. Nat Med 1999; 5: 780-7.
9. Rosenberg, S A, Yang, J C, Schwartzentruber, D J, Hwu, P, Marincola, F M, Topalian, S L, Restifo, N P, Dudley, M E, Schwarz, S L, Spiess, P J, Wunderlich, J R, Parkhurst, M R, Kawakami, Y, Seipp, C A, Einhorn, J H, and White, D E. Immunologic and therapeutic evaluation of a synthetic peptide vaccine for the treatment of patients with metastatic melanoma. Nat Med 1998; 4: 321-7.
10. Schwartzentruber, D J, and al., e. A phase III multi-institutional randomized study of immunization with the gp100: 209-365 217(210M) peptide followed by high dose IL-2 compared with high dose IL-2 alone in patients with metastatic melanoma. J Clin Oncol 2009; 17: 18S.
11. Curran, M A, Montalvo, W, Yagita, H, and Allison, J P. PD-1 and CTLA-4 combination blockade expands infiltrating T cells and reduces regulatory T and myeloid cells within B16 melanoma tumors. Proc Natl Acad Sci USA 2010; 107: 4275-80.
12. Curran, M A, and Allison, J P. Tumor vaccines expressing flt3 ligand synergize with ctla-4 blockade to reject preimplanted tumors. Cancer Res 2009; 69: 7747-55.
13. Rosenberg, S A, Restifo, N P, Yang, J C, Morgan, R A, and Dudley, M E. Adoptive cell transfer: a clinical path to effective cancer immunotherapy. Nat Rev Cancer 2008; 8: 299-308.
14. Hunder, N N, Wallen, H, Cao, J, Hendricks, D W, Reilly, J Z, Rodmyre, R, Jungbluth, A, Gnjatic, S, Thompson, J A, and Yee, C. Treatment of metastatic melanoma with autologous CD4+ T cells against NY-ESO-1. N Engl J Med 2008; 358: 2698-703.
15. Yee, C, Thompson, J A, Byrd, D, Riddell, S R, Roche, P, Celis, E, and Greenberg, P D. Adoptive T cell therapy using antigen-specific CD8+ T cell clones for the treatment of patients with metastatic melanoma: in vivo persistence, migration, and antitumor effect of transferred T cells. Proc Natl Acad Sci USA 2002; 99: 16168-73.
16. Johnson, L A, Morgan, R A, Dudley, M E, Cassard, L, Yang, J C, Hughes, M S, Kammula, U S, Royal, R E, Sherry, R M, Wunderlich, J R, Lee, C C, Restifo, N P, Schwarz, S L, Cogdill, A P, Bishop, R J, Kim, H, Brewer, C C, Rudy, S F, VanWaes, C, Davis, J L, Mathur, A, Ripley, R T, Nathan, D A, Laurencot, C M, and Rosenberg, S A. Gene therapy with human and mouse T-cell receptors mediates cancer regression and targets normal tissues expressing cognate antigen. Blood 2009; 114: 535-46.
17. Morgan, R A, Dudley, M E, Wunderlich, J R, Hughes, M S, Yang, J C, Sherry, R M, Royal, R E, Topalian, S L, Kammula, U S, Restifo, N P, Zheng, Z, Nahvi, A, de Vries, C R, Rogers-Freezer, L J, Mavroukakis, S A, and Rosenberg, S A. Cancer regression in patients after transfer of genetically engineered lymphocytes. Science 2006; 314: 126-9.
18. Hodi, F S, O'Day, S J, McDermott, D F, Weber, R W, Sosman, J A, Haanen, J B, Gonzalez, R, Robert, C, Schadendorf, D, Hassel, J C, Akerley, W, van den Eertwegh, A J, Lutzky, J, Lorigan, P, Vaubel, J M, Linette, G P, Hogg, D, Ottensmeier, C H, Lebbe, C, Peschel, C, Quirt, I, Clark, J I, Wolchok, J D, Weber, J S, Tian, J, Yellin, M J, Nichol, G M, Hoos, A, and Urba, W J. Improved survival with ipilimumab in patients with metastatic melanoma. N Engl J Med 2010; 363: 711-23.
19. Byrne, S M, Aucher, A, Lymperi, S, Alyahya, S H, Dazzi, F, Davis, D M, and Ashton-Rickardt, P G. Cathepsin B Controls the Proliferation but not the Long-Term Self-Renewal of Hematopoeitic Stem Cells. PLoS One 2013; (in press):
20. Byrne, S M, Aucher, A, Alyahya, S H, Elder, M, Olson, S T, Davis, D M, and Ashton-Rickardt, P G. Cathepsin B controls the persistence of memory CD8+ T cells. J Immunol 2012; 189: 1133-43.
21. Beaver, J E, Tasan, M, Gibbons, F D, Tian, W, Hughes, T R, and Roth, F P. FuncBase: a resource for quantitative gene function annotation. Bioinformatics 2010; 26: 1806-7.
22. Finn, R D, Tate, J, Mistry, J, Coggill, P C, Sammut, S J, Hotz, H R, Ceric, G, Forslund, K, Eddy, S R, Sonnhammer, E L, and Bateman, A. The Pfam protein families database. Nucleic Acids Res 2008; 36: D281-8.
23. Barreau, C, Paillard, L, and Osborne, H B. AU-rich elements and associated factors: are there unifying principles? Nucleic Acids Res 2005; 33: 7138-50.
24. Bolognani, F, Contente-Cuomo, T, and Perrone-Bizzozero, N I. Novel recognition motifs and biological functions of the RNA-binding protein HuD revealed by genome-wide identification of its targets. Nucleic Acids Res 2010; 38: 117-30.
25. Altay, G, and Emmert-Streib, F. Structural influence of gene networks on their inference: analysis of C3NET. Biol Direct 2011; 6: 31.
26. de Matos Simoes, R, and Emmert-Streib, F. Bagging statistical network inference from large-scale gene expression data. PLoS ONE 2012; 7(3) e33624.
27. Concepcion, D, Seburn, K L, Wen, G, Frankel, W N, and Hamilton, B A. Mutation rate and predicted phenotypic target sizes in ethylnitrosourea-treated mice. Genetics 2004; 168: 953-9.
28. Komatsu, N, Yamamoto, M, Fujita, H, Miwa, A, Hatake, K, Endo, T, Okano, H, Katsube, T, Fukumaki, Y, Sassa, S, and et al. Establishment and characterization of an erythropoietin-dependent subline, UT-7/Epo, derived from human leukemia cell line, UT-7. Blood 1993; 82: 456-64.
29. Heffner, M, and Fearon, D T. Loss of T cell receptor-induced Bmi-1 in the KLRG1(+)senescent CD8(+) T lymphocyte. Proc Natl Acad Sci USA 2007; 104: 13414-9.
30. Liu, N, Raja, S M, Zazzeroni, F, Metkar, S S, Shah, R, Zhang, M, Wang, Y, Bromme, D, Russin, W A, Lee, J C, Peter, M E, Froelich, C J, Franzoso, G, and Ashton-Rickardt, P G. NFkappaB protects from the lysosomal pathway of cell death. Embo J 2003; 22: 5313-22.
31. Barber, D L, Wherry, E J, Masopust, D, Zhu, B, Allison, J P, Sharpe, A H, Freeman, G J, and Ahmed, R. Restoring function in exhausted CD8 T cells during chronic viral infection. Nature 2006; 439: 682-7.
32. Brooks, D G, Ha, S J, Elsaesser, H, Sharpe, A H, Freeman, G J, and Oldstone, M B. IL-10 and PD-L1 operate through distinct pathways to suppress T-cell activity during persistent viral infection. Proc Natl Acad Sci USA 2008; 105: 20428-33.
33. Tan R, Xu X Ogg G S, Hansasuta P, Dong T, Rostron T, Luzzi G, Conlon C P, Screaton G R, McMichael A J, Rowland-Jones S. 1999. Rapid death of adoptively transferred T cells in acquired immunodeficiency syndrome. Blood, 93 (5), pp. 1506-1510.
34. Brodie S J, Lewinsohn D A, Patterson B K, et al. In vivo migration and function of transferred HIV-1-specific cytotoxic T cells. Nat Med. 1999; 5: 34-41.
35. Gehring, A J, Xue, S A, Ho, Z Z, Teoh, D, Ruedl, C, Chia, A, Koh, S, Lim, S G, Maini, M K, Stauss, H, and Bertoletti, A. Engineering virus-specific T cells that target HBV infected hepatocytes and hepatocellular carcinoma cell lines. J Hepatol 2011; 55: 103-10.
36. Pantoliano M W, Petrella E C, Kwasnoski J D, Lobanov V S, Myslik J, Graf E, Carver T, Asel E, Springer B A, Lane P, Salemme F R; High-density miniaturized thermal shift assays as a general strategy for drug discovery; J Biomol Screen. 2001 December; 6(6):429-40.
37. Dev, A, et al; Erythropoietin-directed erythropoiesis depends on serpin inhibition of erythroblast lysosomal cathepsins; J Exp Med. 2013 Feb. 11; 210(2): 225-232.
38. Zhang, M at al. Serine protease inhibitor 6 protects cytotoxic T cells from self-inflicted injury by ensuring the integrity of cytotoxic granules; Immunity. 2006 April; 24(4):451-61.
39. McCausland, M. M and Crotty, S; Quantitative PCR (QPCR) technique for detecting lymphocytic choriomeningitis virus (LCMV) in vivo; J Virol Methods. 2008 January; 147(1): 167-176.
40. Remington, R., Remington's pharmaceutical sciences. 16th ed. 1980: Mack Pub. Co.
41. Stewart, Solid phase peptide synthesis. 2nd ed. 1984, Rockford: Illinois Pierce Chemical Company.
42. Bodanzsky, B., The practice of peptide synthesis. 1984, New York: Springer Verlag.

1. An isolated polypeptide comprising: (a) the amino acid sequence of SEQ ID NO: 1 or an amino acid sequence having at least 70% identity to SEQ ID NO: 1, or (b) the amino acid sequence of SEQ ID NO: 2, or an amino acid sequence having at least 70% identity to SEQ ID NO: 2.

2. The isolated polypeptide of claim 1, comprising the amino acid sequence of SEQ ID NO: 1, except that amino acid residue 53 is mutated to glycine.

3. The isolated polypeptide of claim 1, comprising the amino acid sequence of SEQ ID NO: 2, except that amino acid residue 50 is mutated to glycine.

4. An expression vector comprising a nucleic acid encoding a polypeptide of claim 1.

5. An in vitro method of increasing the proliferation of a T cell population, comprising transducing at least one T cell in the population with the vector of claim 4.

6. The method of claim 5, wherein the polypeptide encoded by the vector is overexpressed.

7. An isolated T cell containing the expression vector of claim 4.

8. The T cell of claim 7 for use in a method of treating a disease or disorder that would benefit from stimulation of T cell proliferation and/or differentiation.

9. An oligonucleotide that hybridises to a nucleic acid sequence that encodes or controls the expression of the polypeptide according to claim 1 or a nucleic acid sequence that is complementary to a nucleic acid sequence that encodes or controls the expression of the polypeptide according to claim 1.

10. The oligonucleotide of claim 9, wherein the oligonucleotide is from about 5 nt to about 1200 nt, about 6 nt to about 500 nt, about 7 nt to about 100 nt, about 10 nt to about 50 nt length or about 20 nt to about 30 nt in length.

11. An agent which up-regulates the function and/or expression of the polypeptide of claim 1 for use in a method of treating or preventing a disease or disorder that would benefit from stimulation of T cell proliferation and/or differentiation or for use as an adjuvant.

12. The agent of claim 11, wherein the agent is an oligonucleotide that hybridises to a nucleic acid sequence that encodes or controls the expression of the polypeptide or a nucleic acid sequence that is complementary to a nucleic acid sequence that encodes or controls the expression of the polypeptide.

13. The T cell of claim 8 for use in a method of treating or preventing a disease or disorder that would benefit from stimulation of T cell proliferation and/or differentiation or for use as an adjuvant, wherein the disease or disorder is cancer or a viral infection.

14. An agent which down-regulates the function and/or expression of the polypeptide of claim 1, for use in a method of treating or preventing a disease, disorder or condition involving unwanted or excessive proliferation and/or differentiation of T cells.

15. The agent of claim 14, wherein the agent is an oligonucleotide which up-regulates the function and/or expression of the polypeptide for use in a method of treating or preventing a disease or disorder that would benefit from stimulation of T cell proliferation and/or differentiation or for use as an adjuvant.

16. The agent of claim 14, wherein the disease, disorder or condition is an autoimmune disease, an inflammatory disease, Graft versus host disease or transplant rejection.

17. A method of identifying an agent which modulates the binding affinity of the polypeptide of claim 1 for a target, comprising:
i) contacting the polypeptide or a biologically active fragment thereof with the target and a candidate agent;
ii) measuring the affinity of the polypeptide or biologically active portion thereof for the target; and
iii) comparing the affinity of the polypeptide or biologically active fragment thereof for the target in the presence of the candidate agent with the affinity of the polypeptide or biologically active portion thereof for the target in the absence of the candidate compound,
wherein an agent which modulates the binding affinity of the polypeptide is identified if the affinity is increased or decreased in the presence of the agent compared to in the absence of the agent.

18. The method of claim 17, wherein the target comprises an RNA sequence.


SEQUENCE LISTING (6 sequences)

SEQ ID NO: 1 - protein, 418 aa, Homo sapiens
MRESQDAAGAHGWNRVGSTATKWFTGAPFGVQSHRFDISAVYPNWKKFSTFTEAPYSTRYSTQVSHIGPGTYSSKETCFSKKKLMKEVDTGWAKAQEATRLTQLPHFQYQAIMKEKRLKEQKLGPGSYNLKDFLEQLREKPCSTRGLLSSGEVRFRGLTGNYYPGPGNYGEKGNPYTKLEENAWNRSHSEGLMCRMSNKPHPRPYQGSGLGPGTYFFKSDLETYVARSVGTRGPYDTFSGDRSKPLPYGHYSMQKKKPRELMNFKSFVEELNSHHNKKHGVFSKLPRNPKTPTERIYWANLSQCPRTLATSGPSFWLPQEKKCKPVNQPPFLLTSKGSGAKACQMIMGSWNPVGVGRYLNTWLMETKDRRQRYRSLFLSGSKRYLSDLARDMLMQERITPFTKGKCPPTVDYNSDPTP

SEQ ID NO: 2 - protein, 414 aa, Mus musculus
MRPRTHGAPPRNIMSTIPKWFKGAPFGVQSHRFDVSAVYPNQKKFSTFTEAPYSRHHSVELSHIGPGTYNSKDTCFSKKFLEQKLGSGWSQAHEATRLTQLPHFHYQAIKKEKEQQVHKRGPGSYNIKDFITELQKKPQSKRGLLSSGETRFRGFIGNYYPGPGNYGEKGNPYTQLEEKAWNRSHSDGLMCRVSNKPPLFHQGSGLGPGTYTIKSDLETFVKKSTGNRGPYDIFSGERSSPLPYGHYSVQKMKPKELTDYKSFLDEMNSQHKKKQGVFSKYPRDPKHPTERIFWTTLSQCPKNMDIAGPGSWLPHETEQKHVNRPPFLLASKRCGLKAYQMILGTWNPVGVGRYLNTTLMESIDRRQRYRSLYMSEPKRYLQDLTRDRLMQKRITPITKGKCRPTVDYNSDPTP

SEQ ID NO: 3 - DNA, 1257 bp, Homo sapiens
atgagggaaagccaggatgccgccggagctcatggctggaaccgcgtcggctccacggccaccaagtggttcaccggggcgcccttcggggtgcagagccacaggtttgacatctctgctgtttatcccaactggaagaagttcagcaccttcactgaggccccatactccacgcgttattctacccaagtgtcccacataggccctgggacttacagctccaaagagacctgcttcagcaagaagaagctgatgaaggaggtggacacaggctgggccaaggcccaggaagccacgcggctgacccagctaccccacttccagtaccaggccatcatgaaagagaagcggctgaaggagcaaaagctgggccccggctcctacaacctcaaagacttcttagaacagctgcgggagaaaccatgtagcacccgggggctgctcagctctggggaggttcgcttccgaggactcactgggaactactatccaggccctggaaattatggggagaagggtaacccatacaccaagctggaggagaatgcctggaaccggtctcattccgagggcctcatgtgcaggatgagcaacaagccacacccccggccttatcaggggagtggtctgggacccggcacctacttcttcaaaagcgaccttgagacatatgtggcacgatccgtcggcacccgcggcccctatgacactttctctggtgatcggagcaagccactgccttatgggcactactccatgcagaaaaaaaagcccagggaactgatgaatttcaagagctttgtagaagaacttaactcacatcacaataagaagcatggggttttttctaaacttccccgaaacccgaaaacccctacagagaggatttactgggccaacctcagccagtgcccccgcacactggccacatctggccccagtttctggcttccacaagagaagaaatgcaaacccgtcaaccagcccccattcctgttgacctccaaggggtcaggtgcaaaggcctgccagatgattatgggaagctggaacccagtaggtgtgggccgctacctcaacacctggctgatggagacaaaggacaggcggcagcgatatcgatccctattcctgagtggatccaaacgctacctctcagacctggcccgggacatgctcatgcaggaaaggatcacaccatttactaagggaaagtgccctccaactgtggattacaattcagatcctactccttaa

SEQ ID NO: 4 - DNA, 1245 bp, Mus musculus
atgagacctcgcacccacggcgcccccccgcgcaacatcatgtccaccatccccaagtggttcaaaggggcgccctttggggtgcagagccacaggtttgatgtctccgctgtctatcccaaccagaagaaattcagcaccttcacagaggccccatactccagacatcattcggtggaactgtcccacataggacctgggacctataactccaaggatacctgcttcagcaagaagttcctggaacagaagttgggctcaggatggtcccaggcccacgaagccactcggctgacccagctaccccacttccactaccaggccatcaagaaggaaaaagagcagcaggtgcacaagcgtggccctggctcctacaacatcaaagacttcataactgagctgcagaagaaaccacagagcaaacgggggctgctcagctctggggagacacgtttccgaggttttattgggaattattatcctggccctggagattatggggagaaggggaacccgtacacacagctggaggagaaggcctggaaccgctcacattctgacggcctgatgtgtagagtgtctaacaagccacccttgtttcatcagggcagtggcctgggacctggtacctacaccatcaaaagcgatcttgagacctttgtgaaaaagtccactggtaatcgtggcccctatgacattttctctggtgaacggagcagtcctttgccctatggacattactctgtgcagaaaatgaagcccaaggaactgacagattacaagagctttctggacgaaatgaactcacaacacaagaagaaacaaggggttttctcgaaatatccccgagatccgaaacaccccacagagagaattttctggacaacccttagtcagtgccccaaaaatatggatatagctggccctggttcttggcttcctcatgagacggaacagaaacatgtcaaccggccaccgttcctcctggcctccaaacggtgcggcctaaaggcctaccagatgattttgggaacctggaacccagttggcgtaggccgctatctcaacaccacgctgatggagtccatagaccgaaggcagcgataccgttctctgtacatgagtgagcccaagcgatacctgcaagacctaacccgagacagactcatgcagaaacggattacacctattacgaagggcaagtgccgtccaactgtggactacaattcagatcctactccttaa

SEQ ID NO: 5 - DNA, 5259 bp, Artificial Sequence
  Retro protein vector sequence
atgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtgactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttaattgtgagcggataacaattacgagcttcatgcacagtgaaatcatgaaaaatttatttgctttgtgagcggataacaattataatatgtggaattgtgagcgctcacaattccacaacggtttccctctagaaataattttgtttaacttttaggaggtaaaacatatgcatcatcaccaccatcattctagcggcgtcgatttgggcacggagaacttgtattttcaaagcatgcgcgagtctcaggacgcagcgggtgcccacggttggaaccgtgttggttctaccgcgactaaatggtttaccggtgcgccgttcggcgttcagagccaccgctttgacattagcgcggtgtatccgaactggaagaaattctccacctttaccgaggccccgtactccacgcgttacagcacccaagtcagccacattggtccgggcacgtacagcagcaaagaaacctgtttctctaagaagaagctgatgaaagaggtggataccggctgggcgaaagctcaggaagcaacccgcctgacgcagctgccgcactttcaataccaggcaatcatgaaagaaaaacgtctgaaagagcaaaagctgggtccgggtagctacaacctgaaggacttcctggagcagctgcgcgagaagccgtgcagcacccgtggcctgctgagcagcggtgaggtgcgctttcgtggtctgacgggtaactactacccgggtccaggtaactatggcgagaagggtaatccttatacgaaattggaagagaatgcgtggaaccgttcccacagcgagggtctgatgtgccgtatgagcaataaaccgcatcctcgcccgtaccagggtagcggcttgggccctggtacctatttcttcaagagcgacctggaaacgtatgtggctcgtagcgtcggtacgcgtggcccgtacgatacctttagcggtgatcgtagcaaaccgctgccgtatggtcactattctatgcagaagaagaaaccacgcgaactgatgaatttcaaaagcttcgtcgaagagctgaatagccatcacaacaagaagcacggtgtttttagcaagctgccgcgtaacccaaaaaccccgaccgaacgtatttactgggcgaatctgagccagtgcccgcgtactctggccaccagcggtccaagcttctggctgccgcaagaaaagaaatgcaaaccggtgaatcaaccgccgtttctgctgacgtccaaaggctcgggcgcaaaagcgtgtcaaatgatcatgggttcgtggaatccggttggtgttggccgttacctgaatacctggttgatggaaaccaaggaccgccgtcaacgctatcgtagcctgttcttgagcggcagcaaacgttacctgtccgacctggcccgtgatatgctgatgcaggagcgcatcaccccgttcaccaagggcaagtgtccgccgacggttgactacaacagcgatccgacgccgtaactcgagccccaagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaa

SEQ ID NO: 6 - protein, 440 aa, Artificial Sequence
  6xHis-TEV-Retro(1-418)_optEC protein sequence
MHHHHHHSSGVDLGTENLYFQSMRESQDAAGAHGWNRVGSTATKWFTGAPFGVQSHRFDISAVYPNWKKFSTFTEAPYSTRYSTQVSHIGPGTYSSKETCFSKKKLMKEVDTGWAKAQEATRLTQLPHFQYQAIMKEKRLKEQKLGPGSYNLKDFLEQLREKPCSTRGLLSSGEVRFRGLTGNYYPGPGNYGEKGNPYTKLEENAWNRSHSEGLMCRMSNKPHPRPYQGSGLGPGTYFFKSDLETYVARSVGTRGPYDTFSGDRSKPLPYGHYSMQKKKPRELMNFKSFVEELNSHHNKKHGVFSKLPRNPKTPTERIYWANLSQCPRTLATSGPSFWLPQEKKCKPVNQPPFLLTSKGSGAKACQMIMGSWNPVGVGRYLNTWLMETKDRRQRYRSLFLSGSKRYLSDLARDMLMQERITPFTKGKCPPTVDYNSDPTP